(19) United States
(12) Patent Application Publication
(10) Pub. No.: US 2008/0125821 A1
(43) Pub. Date: May 29, 2008

(54) Advanced cardiac life support apparatus and method

(76) Inventor: Gregory Blomquist, Plano, TX (US)

Correspondence Address: Michael Cameron, 2025 Savannah Drive, McKinney, TX, 75070, US

(21) Appl. No.: 11/593,917

(22) Filed: Nov. 7, 2006

(51) Int. Cl. A61N 1/00; A61H 31/00

(52) U.S. Cl. 607/6; 601/41; 607/5

(57) ABSTRACT

An apparatus (300, 400) and method operable to guide responders, via audible prompts and visual cues, through the proper procedures to be applied to a patient during a cardiac arrest. An embodiment of the present invention advantageously makes the ACLS procedures, such as types and dosages of medications to administer, and sequence of performing actions (such as cardiac pulmonary resuscitation (CPR)) on the patient easier to acknowledge and follow. An exemplary embodiment of the present invention (300) can be located on a conventional hospital crash cart which stores the emergency equipment and medications, or can be hardware and application software, the application software being loadable and loaded into computer hardware (200).

### CROSS REFERENCE TO RELATED APPLICATIONS

This application claims the benefit of U.S. Provisional Application No. 60/35,371 filed on Nov. 12, 2005, entitled “THE CODE BLUE CLOCK.”

### TECHNICAL FIELD

The preferred embodiment of the present invention relates to a computer hardware and software based system used during the performance of cardiac life support procedures.

### BACKGROUND

Each year thousands of patients suffer sudden cardiac death. Since 1919 cardiovascular disease (CVD) has been the number one killer in this country. Almost 40% of the deaths in this country are related to CVD in some form. Further, the population is aging resulting in more people being admitted to the hospitals. When a sudden cardiac event is discovered to have occurred to a patient in a hospital, a series of actions take place in an attempt to resuscitate that person. Optimally, a response team (referred to conventionally as a code blue response team) consisting of nurses, supervisors, respiratory personnel, technicians and a physician who, preferably, is well-versed in advanced cardiac life support (ACLS) procedures, is summoned to undertake the resuscitation procedures. However, many times, a physician is either not immediately available to coordinate the ACLS or is not experienced with the specific protocols.

Most nursing personnel who work on a typical hospital floor are not required to learn or be certified in ACLS. Furthermore, most physicians outside of an intensive care unit (ICU) or emergency room (ER) do not use the ACLS protocols often enough to retain the precise knowledge of the many medications and specific timing sequences involved in a code blue situation.

An unfortunate, but very typical situation is as follows: A nurse goes in to check on a patient during 3:00 a.m. rounds. The nurse notices that the patient's breathing is extremely shallow and the patient's heart rate is only ten beats per minute. The patient is not awakened to the voice of or shaking by the nurse. The nurse activates a switch (conventionally known as a code blue button) located in the patient's room, for example, on the wall, which sends out an urgent notification to all necessary responders within the hospital, except a physician. Often, there may be only one physician in the entire building at that hour, and if this physician is treating critical patients in the ER he/she cannot leave that area. The floor secretary would then notify the patient's private doctor via an answering service. The answering service typically assures the floor secretary that if the doctor doesn't call back in 20 minutes they will notify the doctor again. Meanwhile, the patient may stop breathing. A responder who remembers the ACLS procedure will be critically needed in the patient's room. However, it is often the case that no one in the room works in the ER or has kept up with the latest changes in the ACLS protocols. As a result, the patient may die, notwithstanding the heroic efforts of the responders who, unfortunately have inadequate knowledge and experience of ACLS.

What is desired is a method and apparatus operable to guide responders, via audible prompts and visual cues, through the medications, dosages and proper timing sequences required during a cardiac arrest event, according to the ACLS protocols.

### SUMMARY

A preferred embodiment of the present invention comprises an ACLS method and apparatus operable to guide responders, via audible prompts and visual cues, through the proper procedures to be applied to a patient during a cardiac arrest. A preferred embodiment of the present invention can be located on a conventional hospital crash cart which stores the emergency equipment and medications, or can be hardware and application software, the application software being loadable and loaded into computer hardware having a central processing unit (CPU), memory, input/output means such as a keyboard, mouse, touchpad, trackball, speaker, buzzer, illuminated indicators, video display and the like, data busses and operating software and drivers. The computer hardware can be a mainframe, personal computer (PC), terminal, tablet PC, laptop PC, personal digital assistant (PDA), and the like. Input to the computer hardware/software can be facilitated via keystrokes, screen taps, button or switch activation, voice recognition, mouse clicks, biometric recognition and the like. A preferred embodiment of the present invention advantageously makes the ACLS procedures, such as types and dosages of medications to administer, and sequence of performing actions (such as cardiac pulmonary resuscitation (CPR)) on the patient easier to acknowledge and follow.

To those skilled in the art to which this invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

### BRIEF DESCRIPTION OF THE DRAWINGS

For a better understanding of the preferred embodiments of the present invention including the features, advantages and specific embodiments, reference is made to the following detailed description along with accompanying drawings in which:

FIGS. 1A-1TTT provide a flow chart of an exemplary method of the present invention as would be administered to an adult patient;

FIG. 2 is a block diagram of a computer based exemplary apparatus adapted to perform methods of the present invention;

FIGS. 3A-3C are three views of an exemplary apparatus adapted to perform the methods of the present invention adapted to be installed on a hospital crash cart; and

FIG. 4 is an exemplary apparatus adapted to perform the methods of the present invention in the form of a PDA.

References in the detailed description correspond to like references in the Figures unless otherwise noted. Like numerals refer to like parts throughout the various Figures.

### DETAILED DESCRIPTION OF A PREFERRED EMBODIMENT

While the making and using of the preferred embodiments of the present invention is discussed in detail below, it should be appreciated that the preferred embodiments of the present invention provides many applicable inventive concepts which can be embodied in a wide variety of specific contexts. Some features of the preferred embodiment shown and discussed may be simplified or exaggerated for illustrating the principles of the invention.

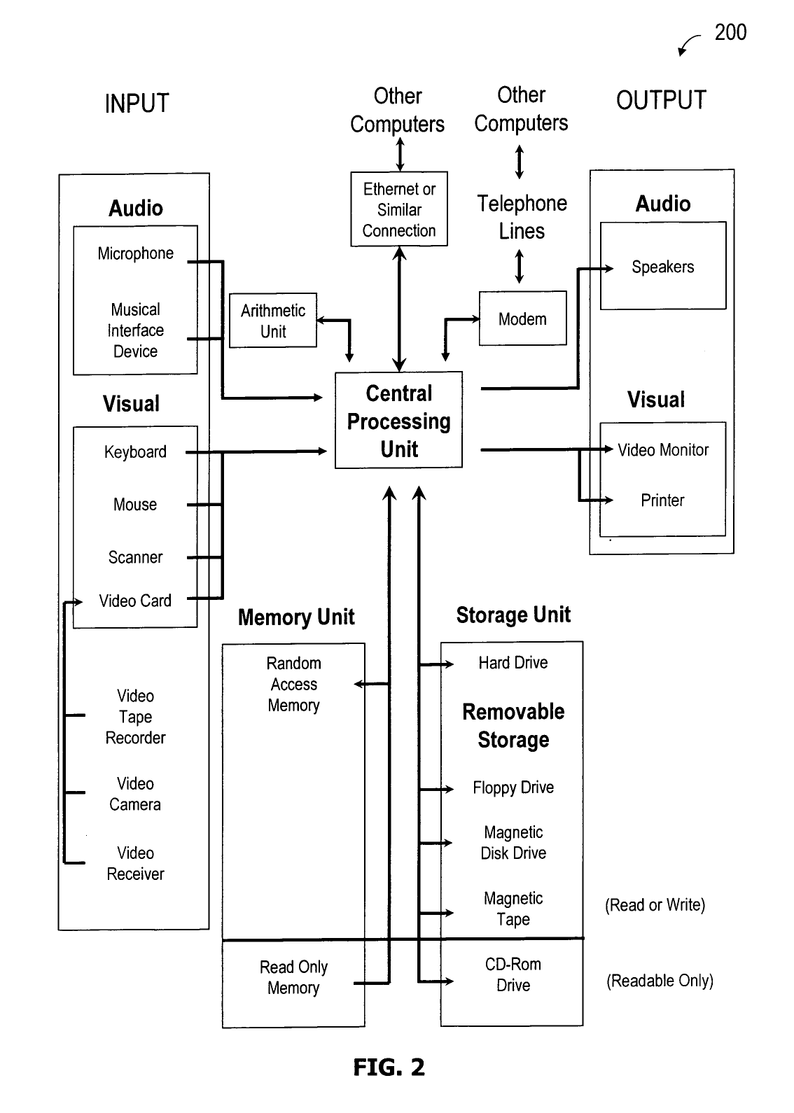

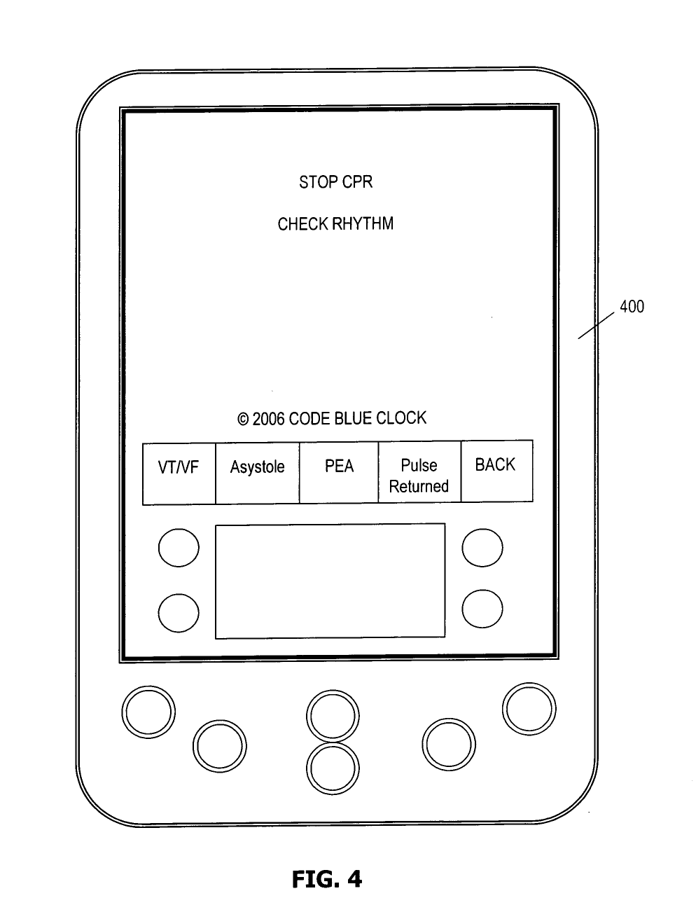

The preferred embodiment of the present invention comprises an ACLS method and apparatus operable to guide responders, via audible prompts and visual cues, through the proper procedures to be applied to a patient during a cardiac arrest. As seen in FIG. 2, a preferred embodiment of the present invention preferably comprises application software run on computer hardware, the application software being loadable and loaded into computer hardware having a central processing unit (CPU), memory, input/output means such as a keyboard, mouse, touchpad, touch screen trackball, speaker, buzzer, illuminated indicators, printer, video display, ports (such as serial, parallel, USB, infrared (IR), and wireless) and the like, data busses, a power supply and regulation circuit, and operating software and drivers. The computer hardware can be a mainframe, personal computer (PC), mobile terminal, thin client terminal, tablet PC, laptop PC, personal digital assistant (PDA) (as seen in FIG. 4), and the like. Input to the computer hardware/software can be facilitated via keystrokes, screen taps, button or switch activation, voice recognition, mouse clicks, biometric recognition and the like.

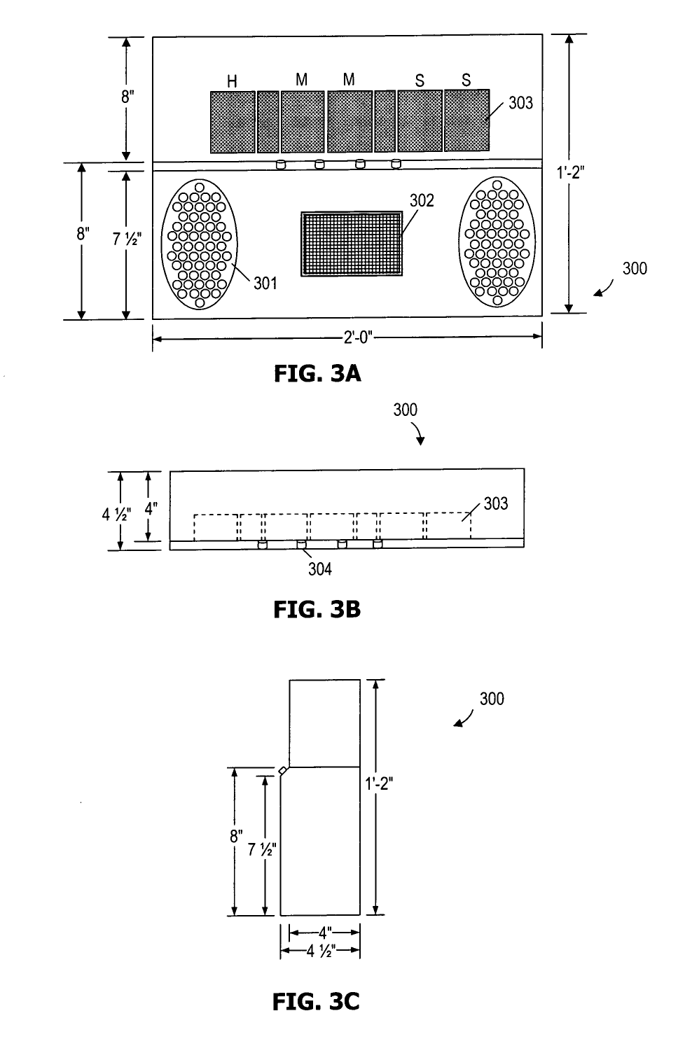

An embodiment of the present invention is in the form of at least one computer readable medium and processor instructions contained on the at least one computer readable medium. The processor instructions contain the algorithms to be used during ACLS procedures, such algorithms generally in the form of decision trees. A decision tree is a set of decisions and their possible consequences used to create a plan to reach an objective, in this case the resuscitation of a patient. A decision tree is a predictive model that is a mapping of observations about a patient to conclusions about the patient's eventual outcome. The processor instructions are configured to be readable from the at least one computer readable medium by at least one processor and thereby cause the at least one processor to determine when to provide prompts, accept inputs, start timers, and indicate the status of events. An embodiment of the present invention 300 can be located on a conventional hospital crash cart which stores the emergency equipment and medications as seen in FIGS. 3A-3C. FIG. 3A is a front view of apparatus 300 having audio output 301, touch sensitive digital display 302 and digital clock displays 303. FIG. 3B is a top view showing digitals clock displays 303 from the top and input buttons 304 for responding to prompts and commands of the apparatus 300. FIG. 3C is a side view of apparatus 300.

Generally speaking, a preferred embodiment of the present invention comprises an apparatus for use in performing ACLS procedures having at least one prompting means for obtaining information from a user; at least one input means for obtaining user responses to information prompts; at least one timing means for obtaining timing information based on elapsed time or the temporal occurrence of events; a logic means operable to determine a decision path selection based on user responses to information prompts and timing information; and at least one output means for providing communication cues to guide responders through the proper procedures to be applied to a patient during a cardiac arrest based on output from the logic means. The operation of the computer hardware, being directed via the software, can be changed by upgrading or modifying the software. Such changes can be made to the software, via a hardware port, when, for example, ACLS procedures are improved or changed or as new medications and dosages are introduced. The scope of the present invention is meant to at least encompass the ability to make such changes. The preferred embodiment of the present invention advantageously makes the ACLS procedures, such as types and dosages of medications to administer, and sequence of performing actions (such as cardiac pulmonary resuscitation (CPR)) on the patient easier to acknowledge and follow.

The following will generally describe the operation of a preferred embodiment of the present invention. The details of the method and the apparatus which implements the method will follow the general discussion. Assume that a hospital patient is undergoing cardiac arrest and a physician well versed in the ACLS procedures is not readily available. A nurse wheels a crash cart including a preferred embodiment of the present invention into the patient's room. The apparatus is activated by means of an on-off switch. In one embodiment of the present invention, a touch screen prompts the nurse to initiate a specific algorithm by choosing whether the patient is an adult or child, and within seconds the first audible command is issued from the apparatus at a volume that is suitable for the responders to hear. Once the first command is followed, the nurse then touches the screen to inform the apparatus of such fact, and a first timer of a plurality of timers, is activated. The first timer counts down to a certain level (such as zero), after which an addition command is given. Additional timers, e.g., a second timer, third timer, fourth timer, of the plurality of timers operate concurrently and/or consecutively, each providing a precise determination of when an addition event in the ACLS procedure is to occur or an action to be commanded. Such actions and events can include, among other things, the dosage and administration of medications, performance of CPR, and the monitoring of patient vital signs. If the patient's conditions changes during the process of resuscitation, the method and apparatus is adapted to prompts the nurse for the change of condition and the method causes the screen selections to change accordingly. Alternatively, the scenario could involve a physician who is not well-versed in ACLS procedures using a PDA that has been loaded with application software that is adapted to perform the methods of a preferred embodiment of the present invention. In either case, the audible and visual prompts can be in English or in any other language which is best suited to the location and language of the responders.

As can be seen, a preferred embodiment of the present invention is adapted to guide responders through a difficult situation by instructing them in what to do in sequential order and by timing the events with precision. In this manner, proper medications in the correct dosages are administered and actions are taken at the proper time, thus improving the probability of survival of the patient. During the entire resuscitation process a preferred embodiment of the present invention is providing audible prompts to the responders about the timely nature of administering medicine and at what intervals.

An additional aspect of a preferred embodiment of the present invention is the use of medications in color-coded packages medications in combination with the apparatus and method of the present invention. In this way, the audible commands can be simplified to directing a nurse to administer a medication in a particular package, without regard to the medical name of medication. In this manner, any responder who can start an intravenous (IV) fluid can administer the proper medications as the situation warrants.

A further aspect of a preferred embodiment of the present invention is in combination with an automatic external defibrillator (AED). Such combination could, for example, be implemented using the software and hardware components of a preferred embodiment of the present invention and the power source of the AED. The use of a preferred embodiment of the present invention with an AED would result in a modification to the method of a preferred embodiment of the present invention as the AED, not the responder, will recognize the individual rhythms before proceeding with the protocols. In other words, the protocols will remain intact except for the steps which request rhythm recognition. It is noted that drug protocols, IV and airway prompts and CPR cycles are not part of the AED. Another aspect of a preferred embodiment of the present invention relates to a safety issue. Preferably, when the responder is prompted to select one of a choice of drugs, the choices, while appearing on the same screen, are located at a proximate distance apart so as to avoid inadvertently selecting the wrong choice. In a further safety aspect, for especially critical decisions, confirming prompts may be provided to confirm the choice of the responder. Such confirmation can also be made by using a bar code or similar reader to read bar codes from medicine packaging to confirm steps that have been taken. In a further aspect of a preferred embodiment of the present invention, a list of the actions taken are recorded within the memory portions of the hardware, and further are displayed on the video display for quick reference during the ACLS process. Further, an option can be provided to the user, via the video display or via a separate switch or key stroke, to return to a previous display to review actions taken or the choices that were available. The actions stored in memory can be printed at a printer concurrently as the events are occurring or at a later time. Such actions can be correlated to the actual time such events occurred, such time being a universal worldwide time (UTC) or the local time. For example: IV access obtained at 2:34; First epinephrine at 4:10; Second shock at 7:42, etc. If a mistake is made during the ACLS process, the apparatus will allow the responder to note such mistake, and any actions taken in response thereto, via a keyboard.

If it becomes necessary to pronounce the patient dead, the apparatus of a preferred embodiment of the present invention will accept an input from the responder of the pronouncement, whereupon the apparatus will provide a prompt to be sure this is the desired action.

A preferred embodiment of the present invention, as implemented, will be adapted to receive input from the responder concerning the age, weight and/or height of the patient. In response to the input, the apparatus will be adapted to tailor the correct dosages for the medications and level of voltage shocks to be applied. For example, the video display and audible prompt may provide: “If newborn go to step x, infant go to step y, child go to step z”. In this manner, a look-up table loaded in the memory computer hardware would be have values representing dosage data for newborns, toddlers, small children and children. The same concept is extended to, for example, IV and endotracheal tube sizes. This would advantageously overcome the need to make such calculations during the ACLS procedure.

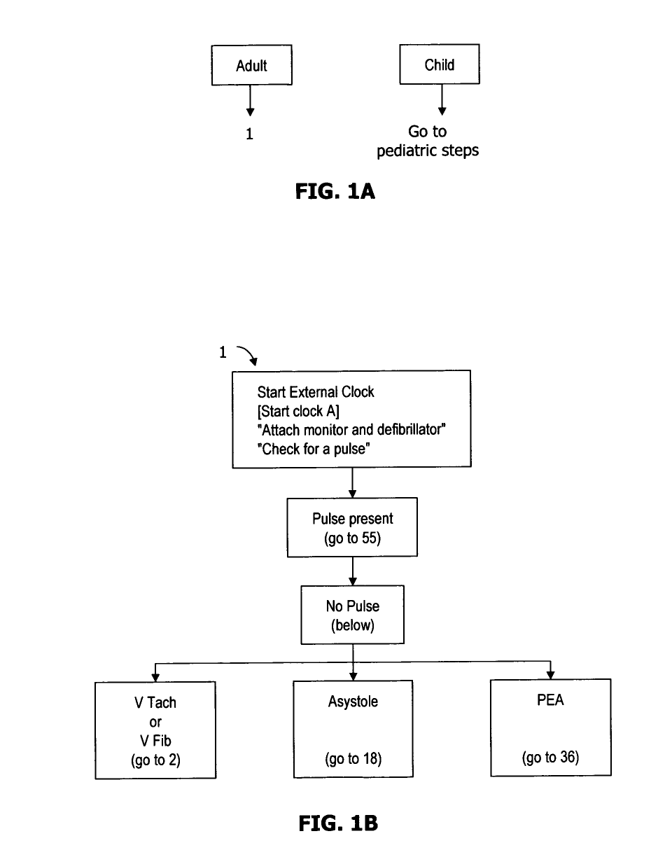

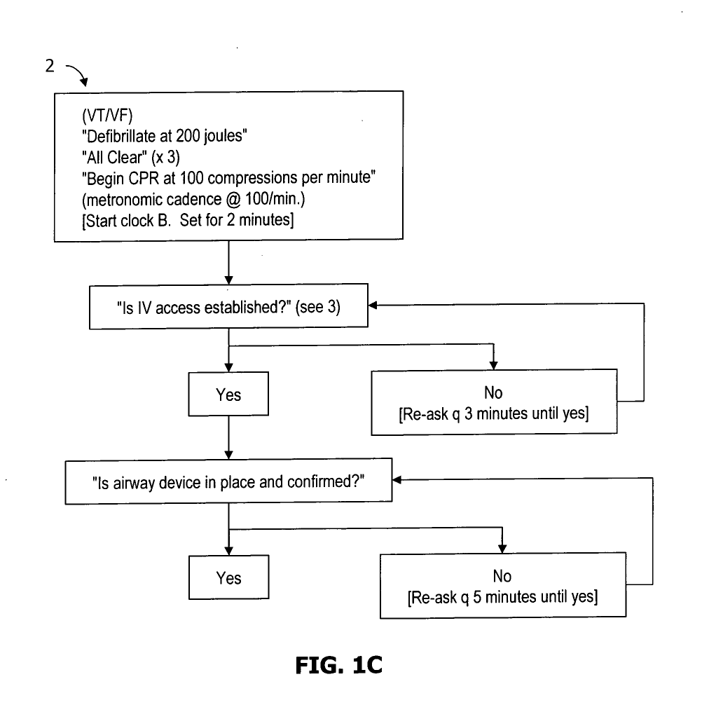

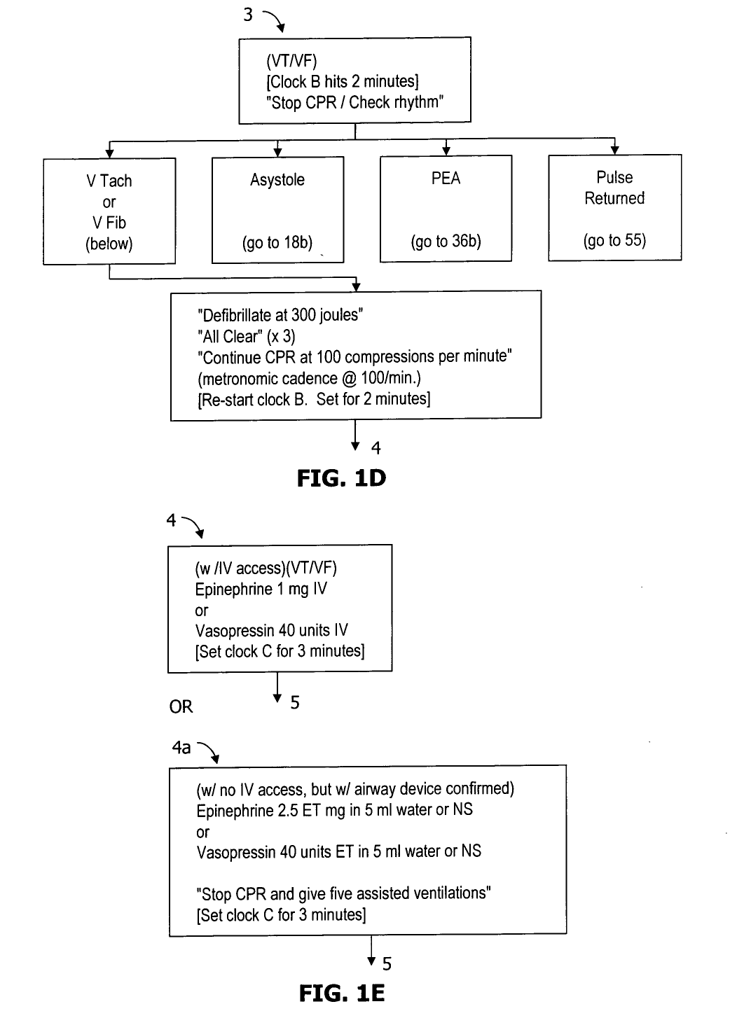

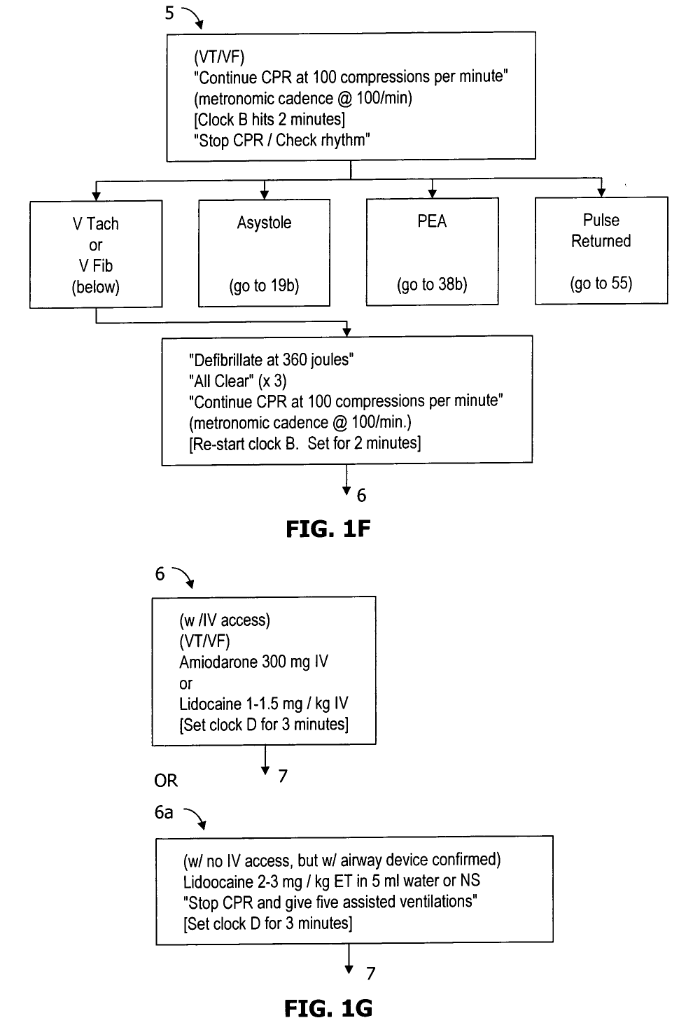

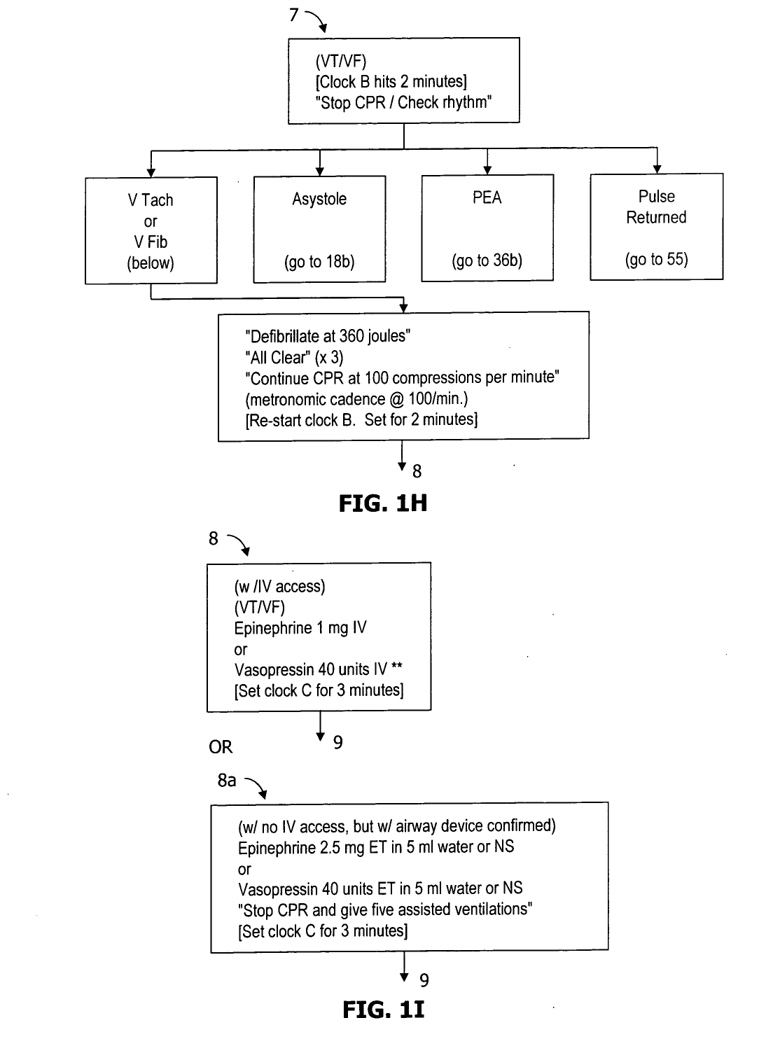

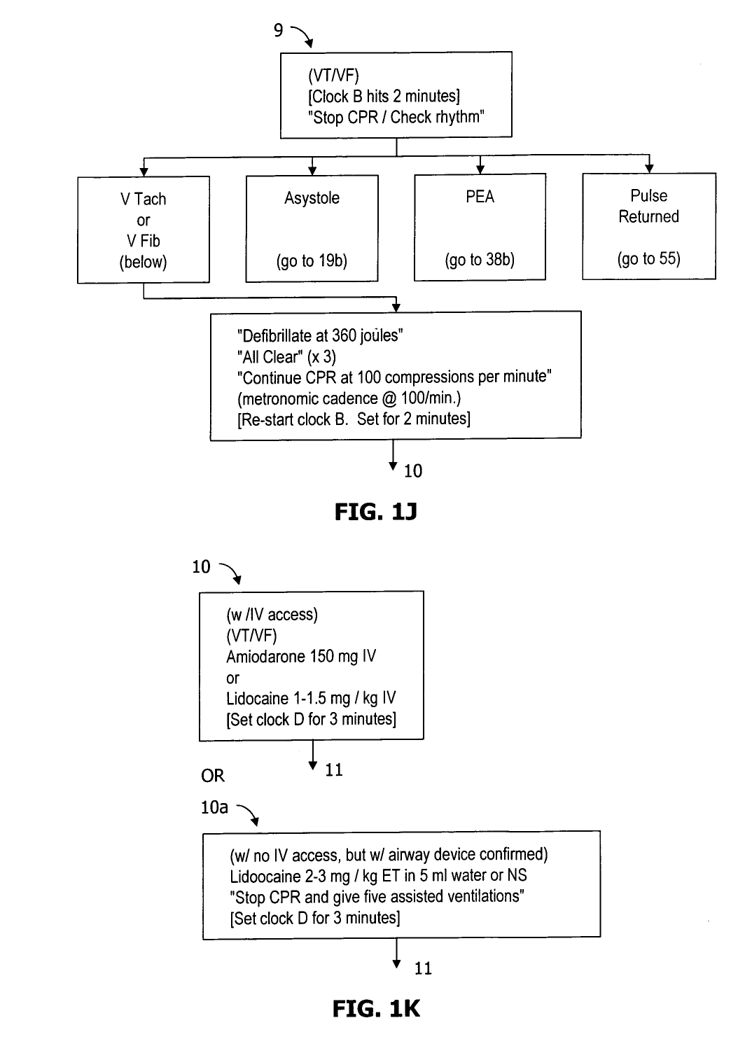

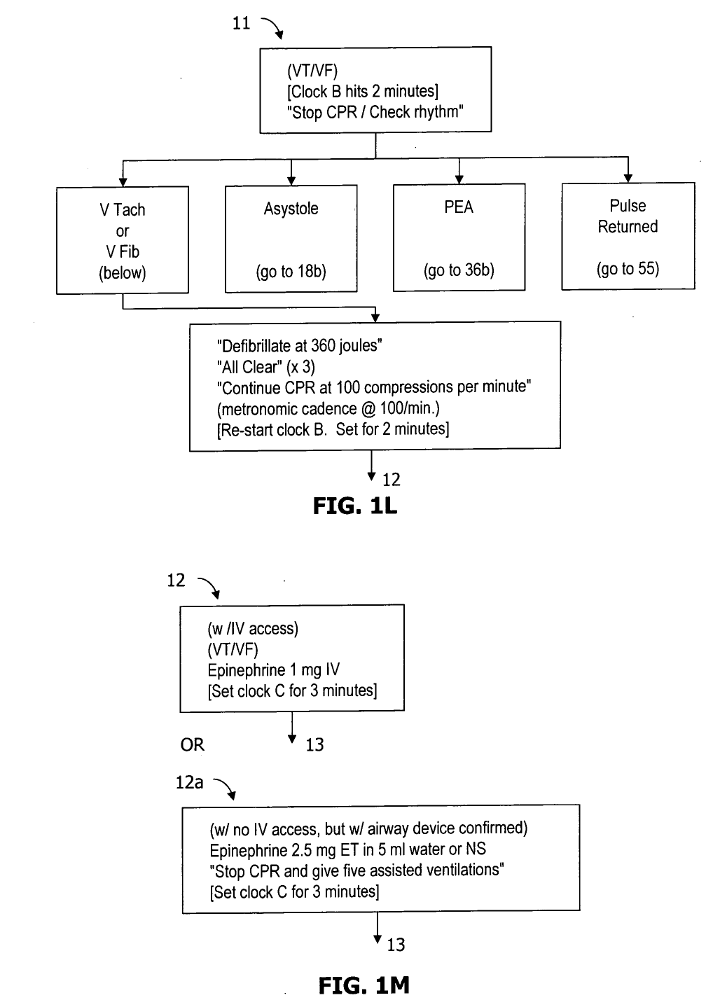

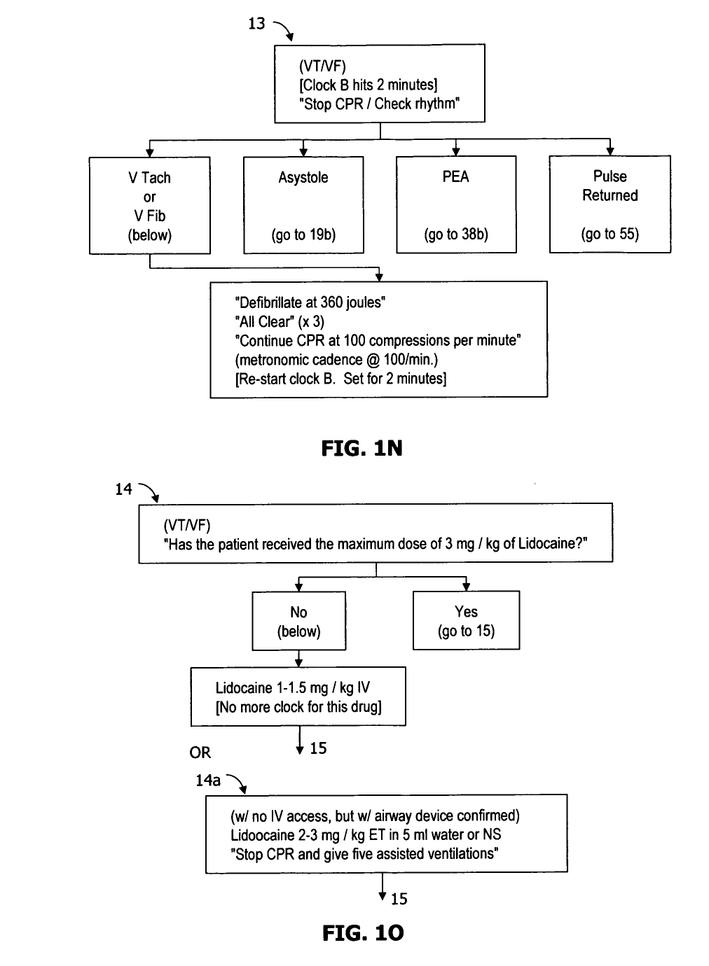

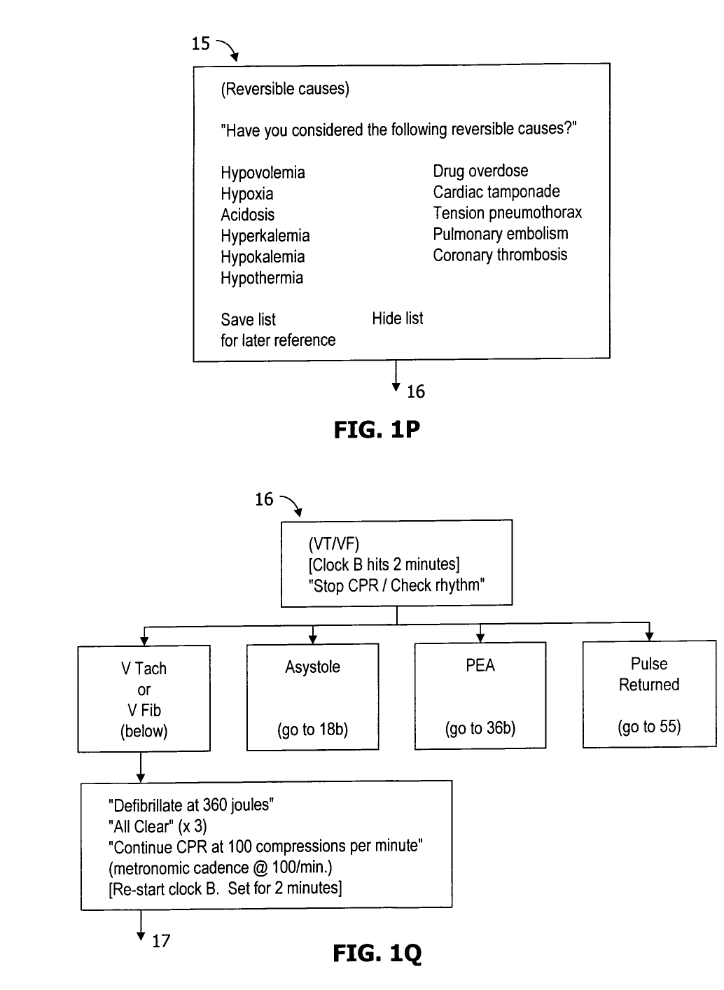

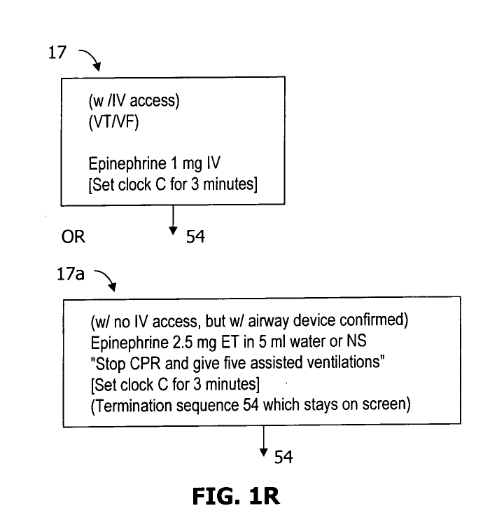

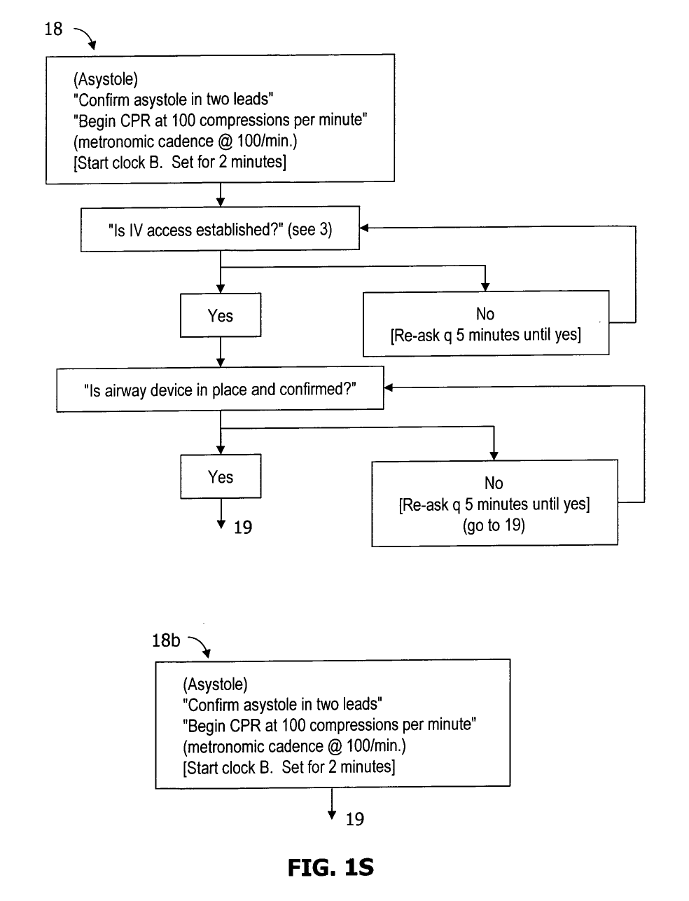

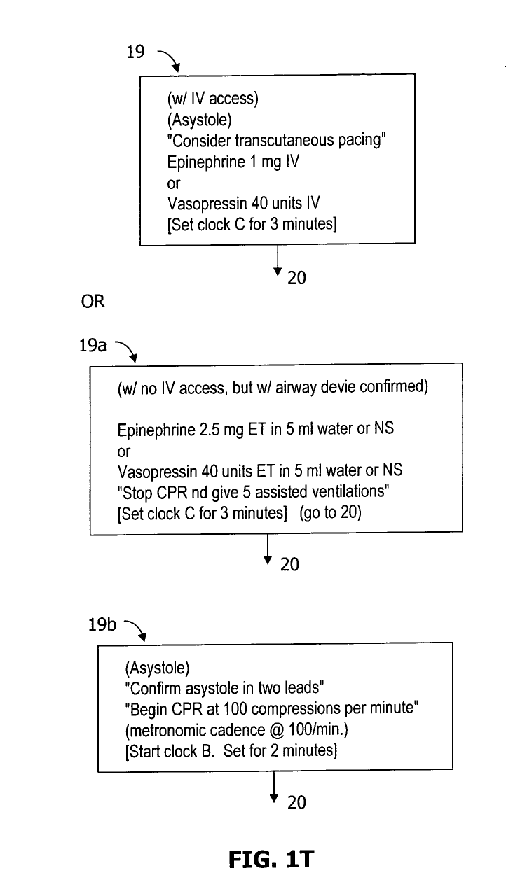

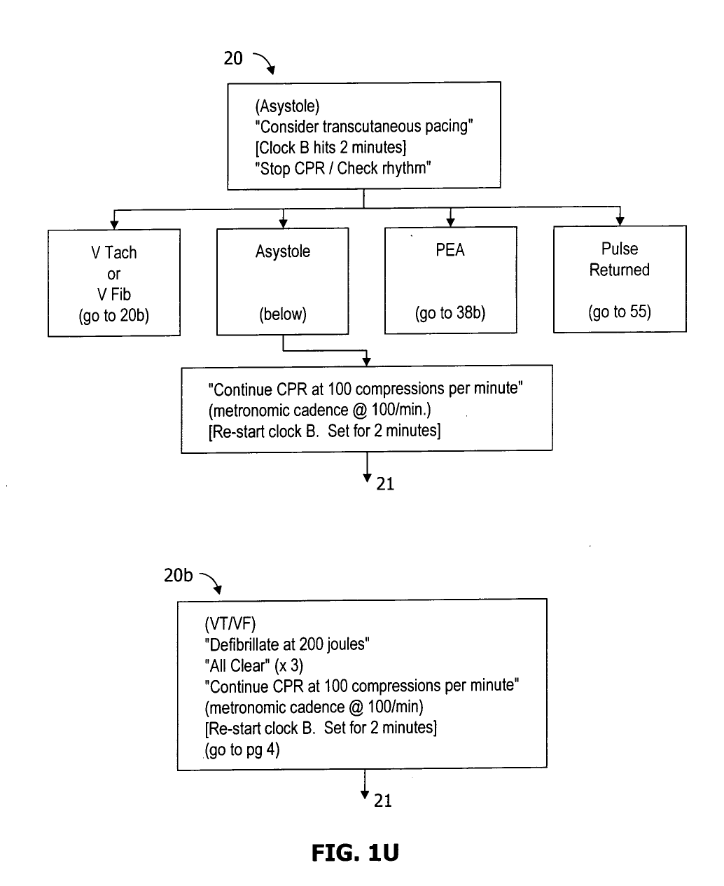

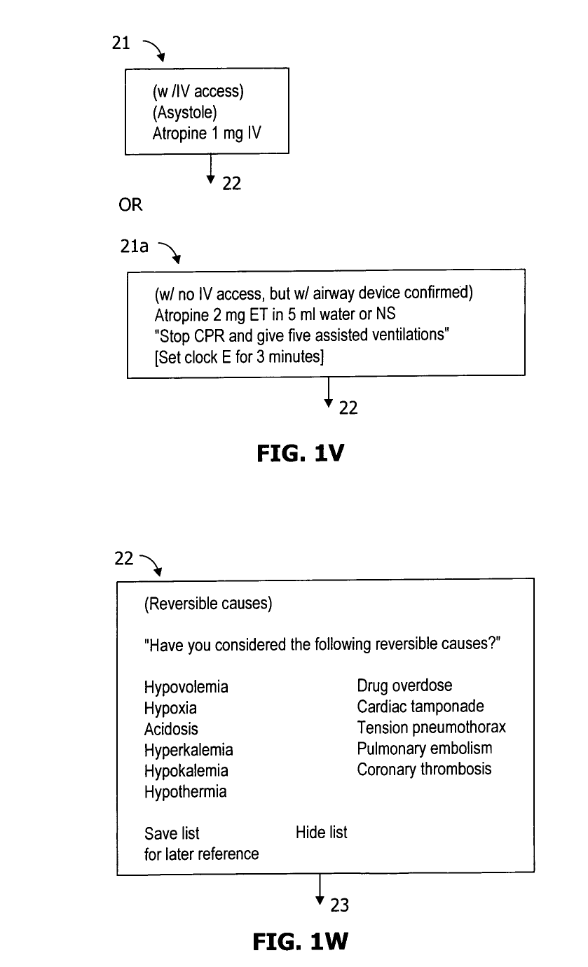

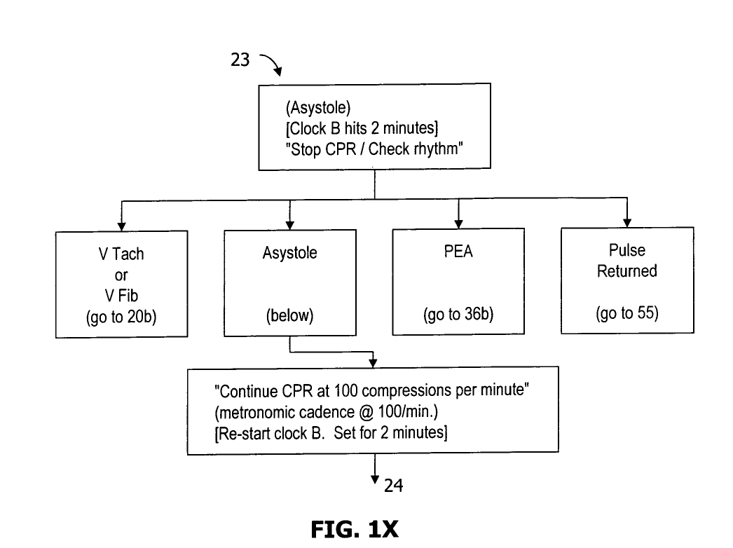

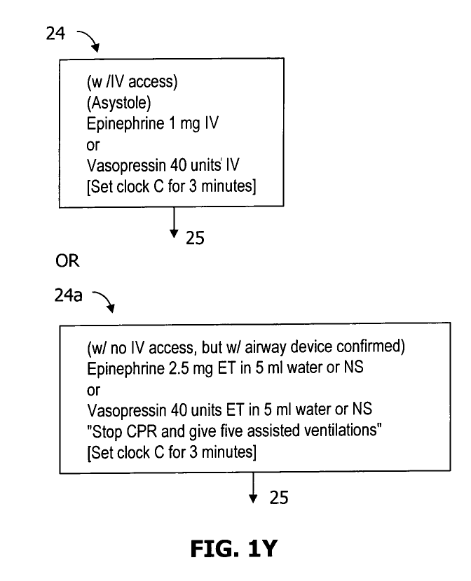

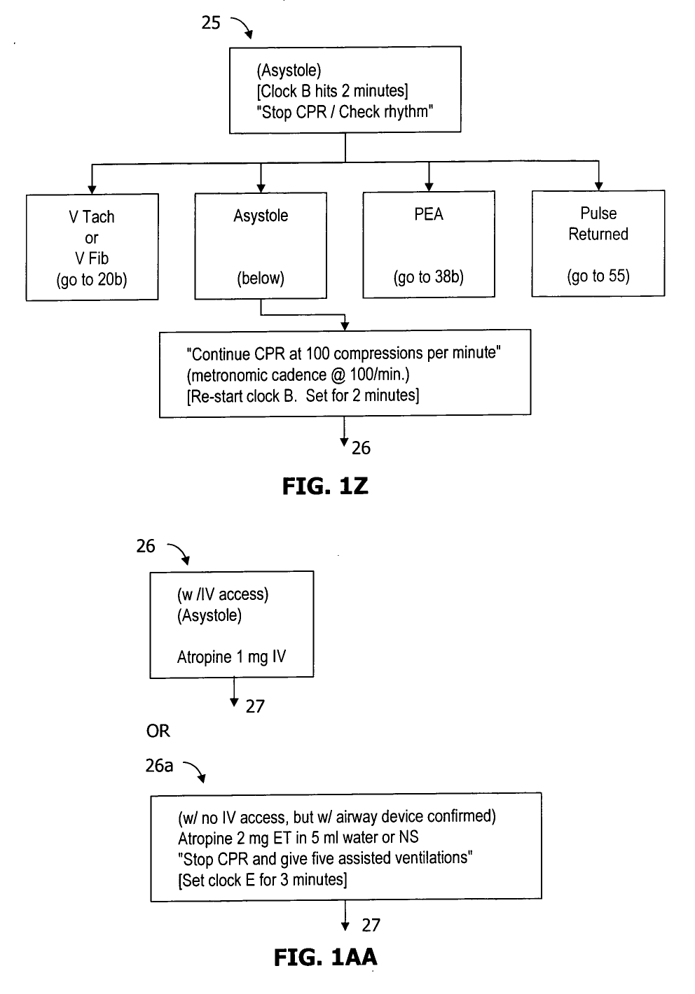

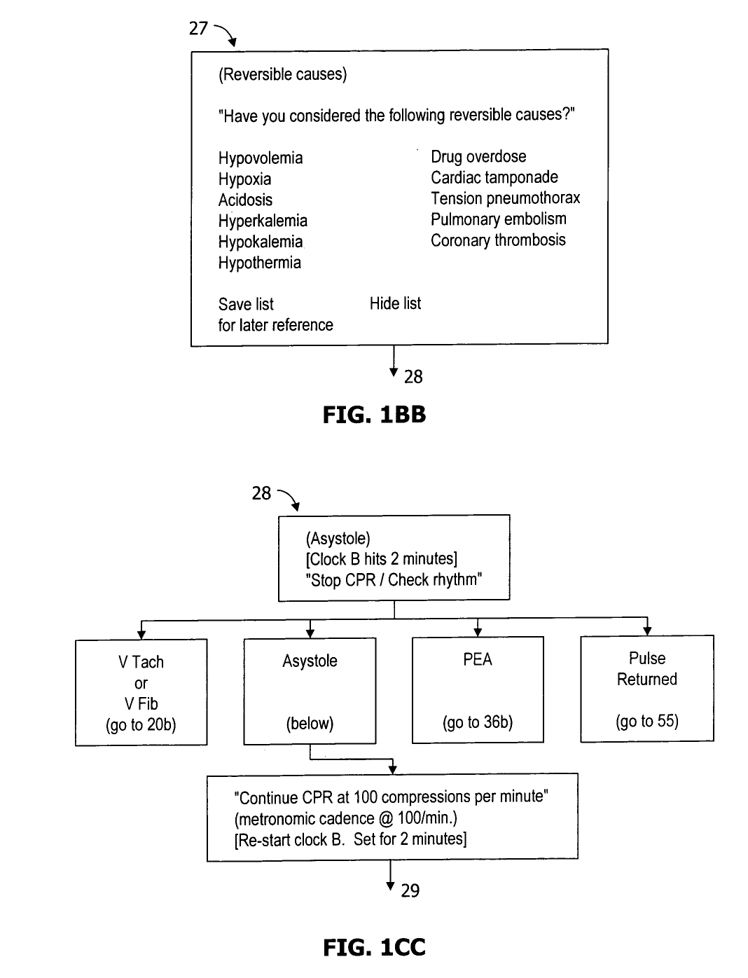

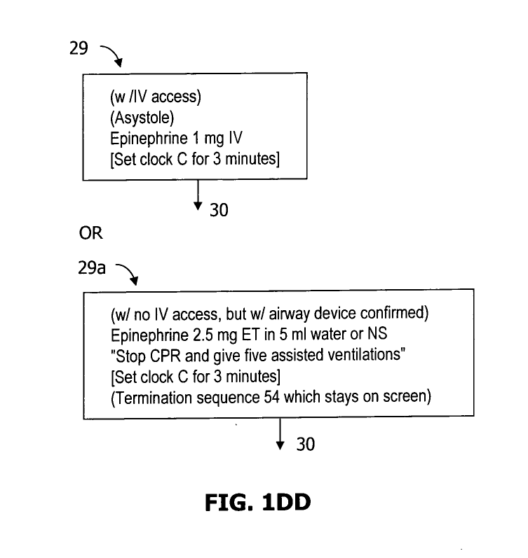

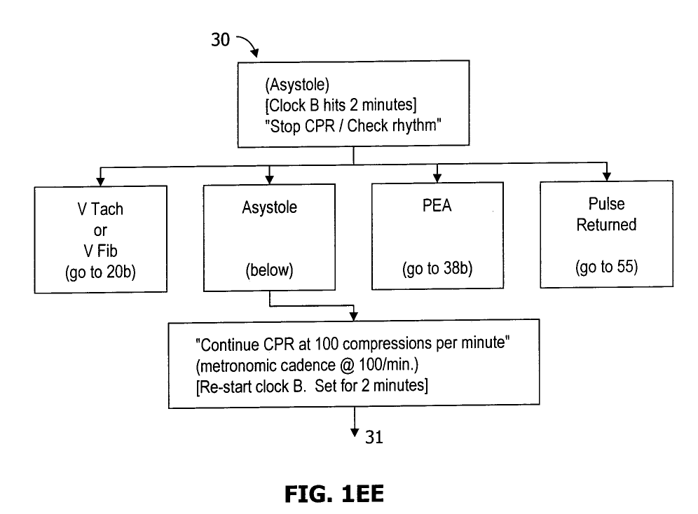

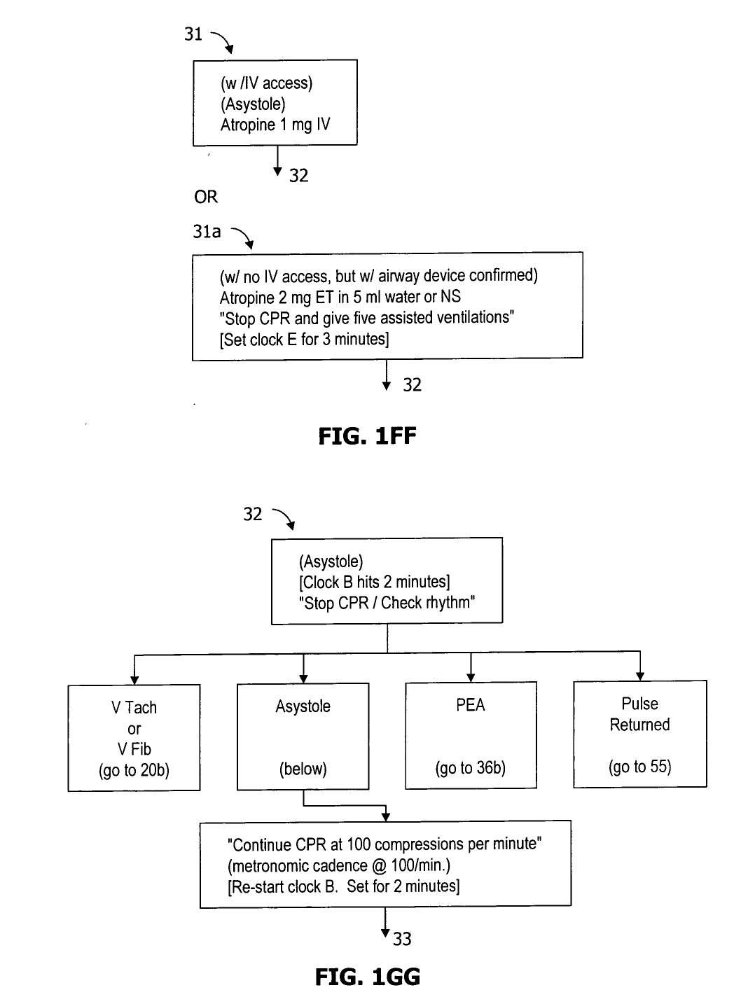

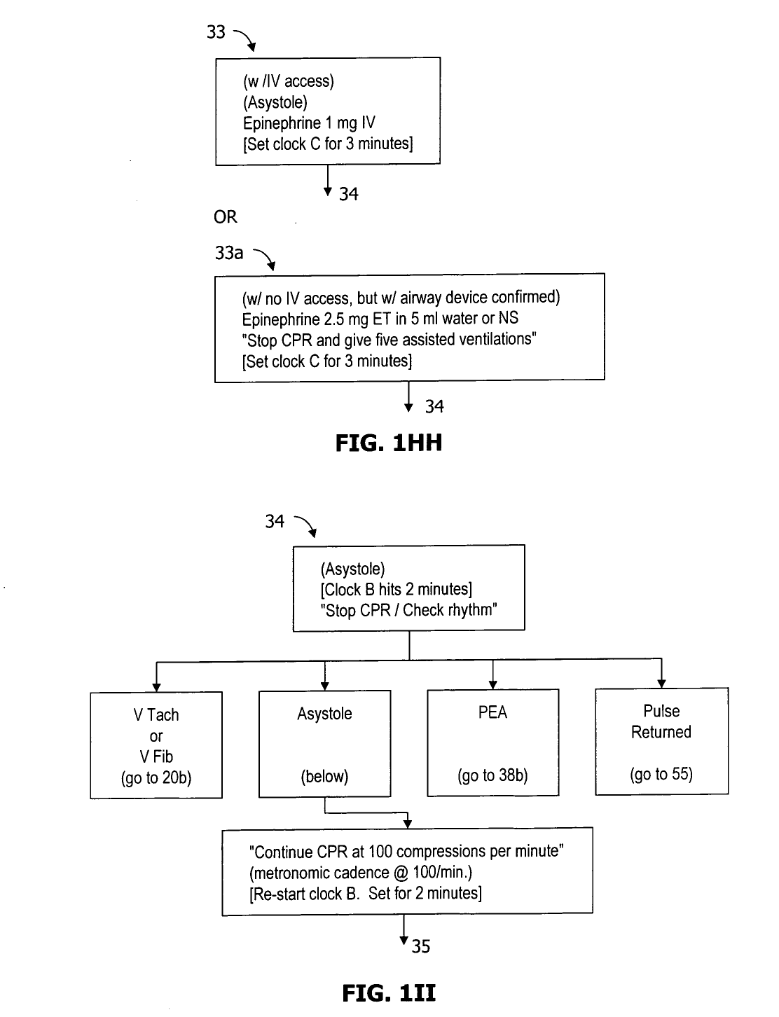

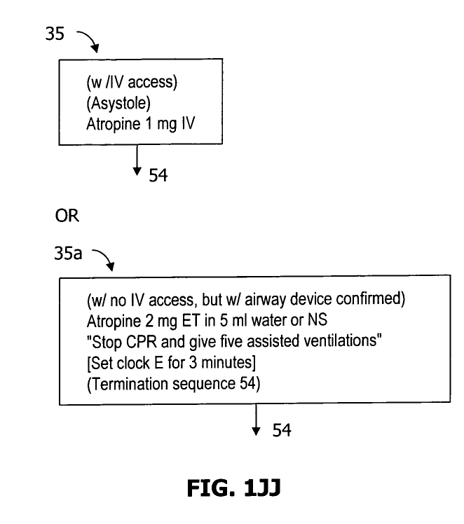

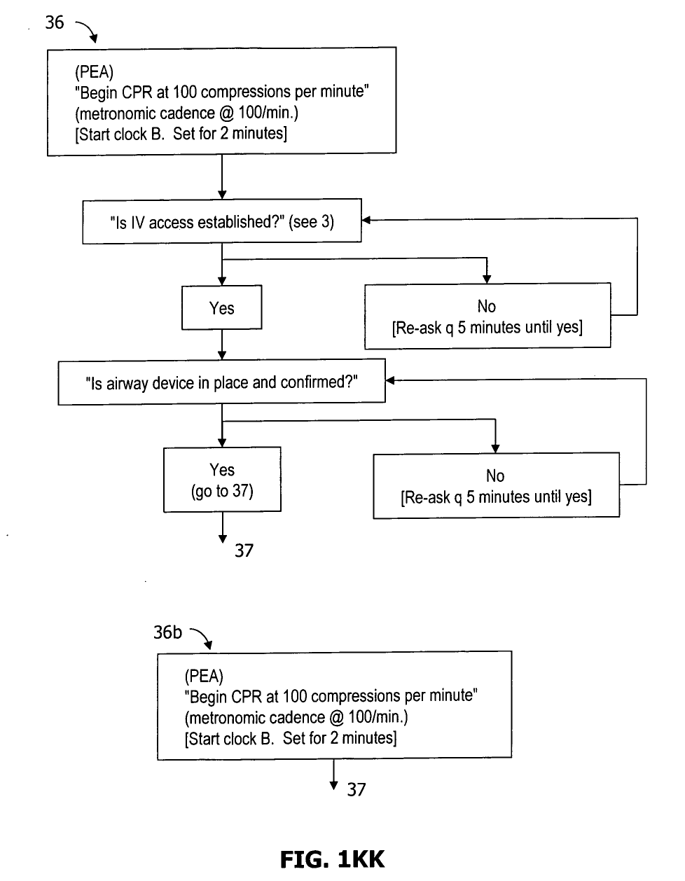

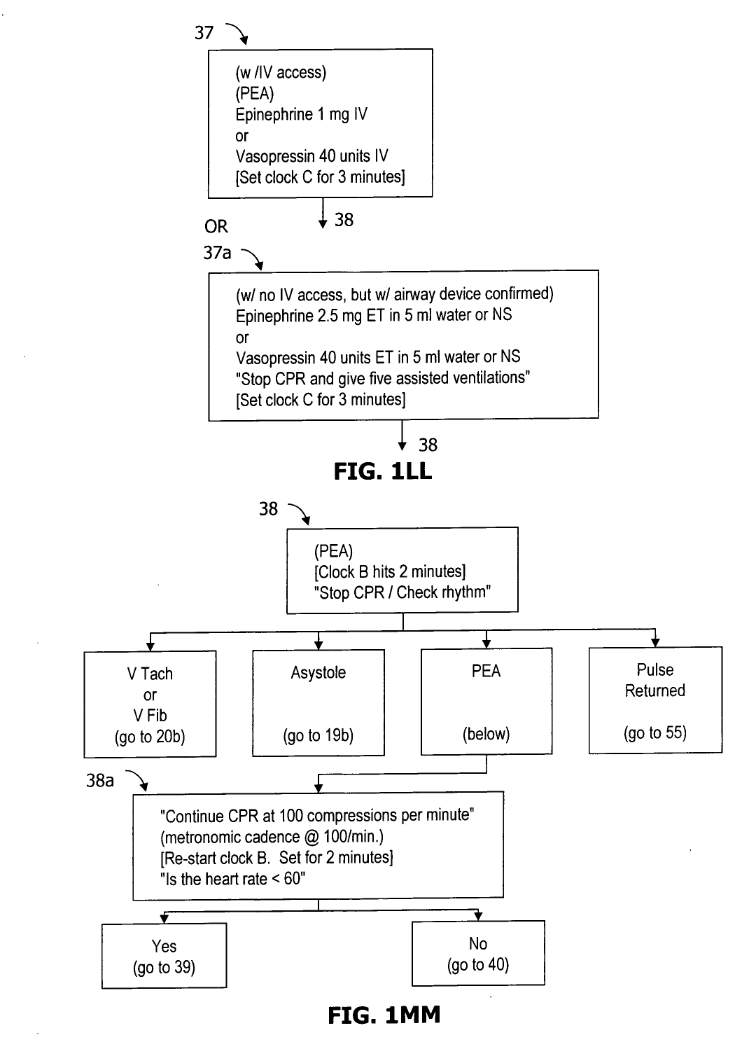

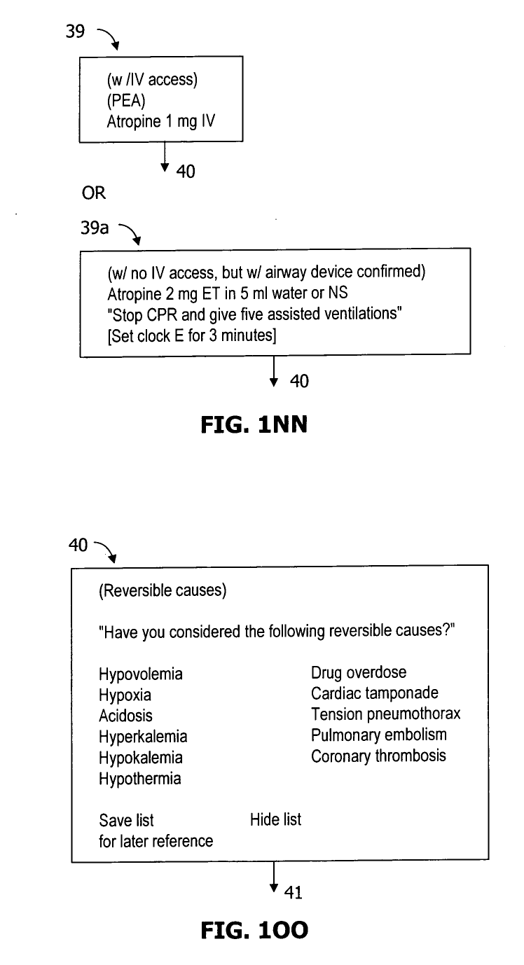

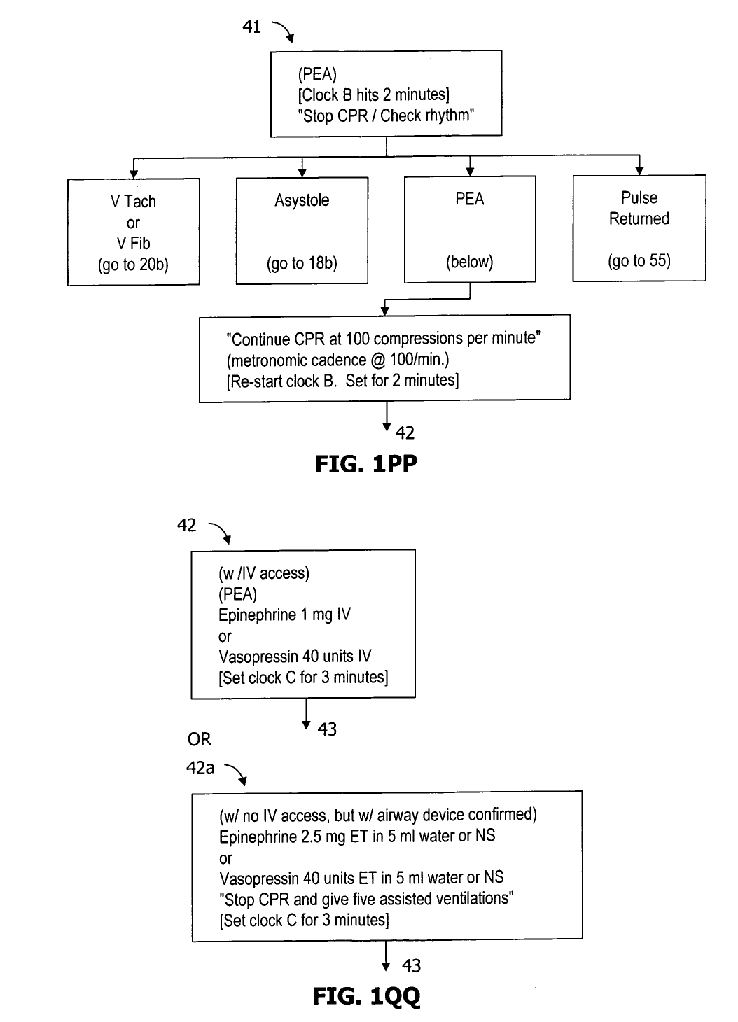

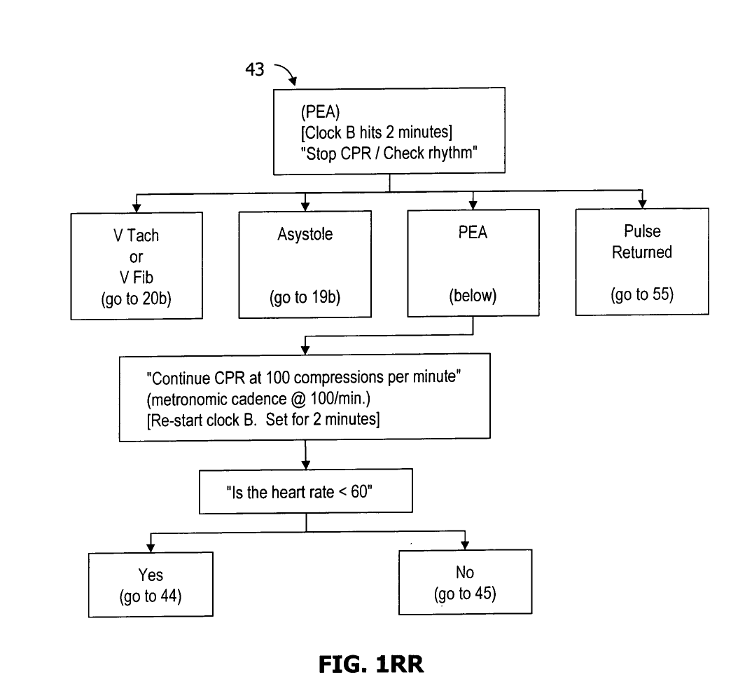

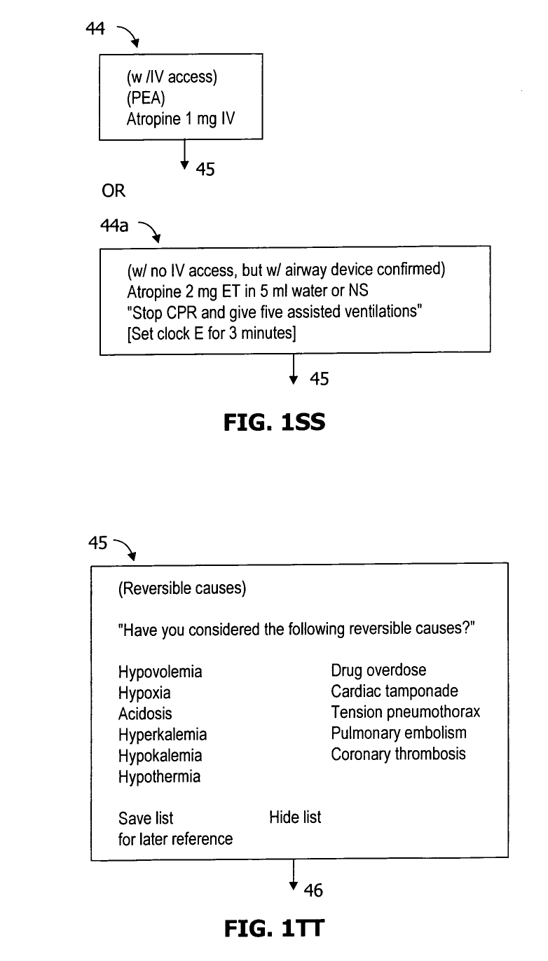

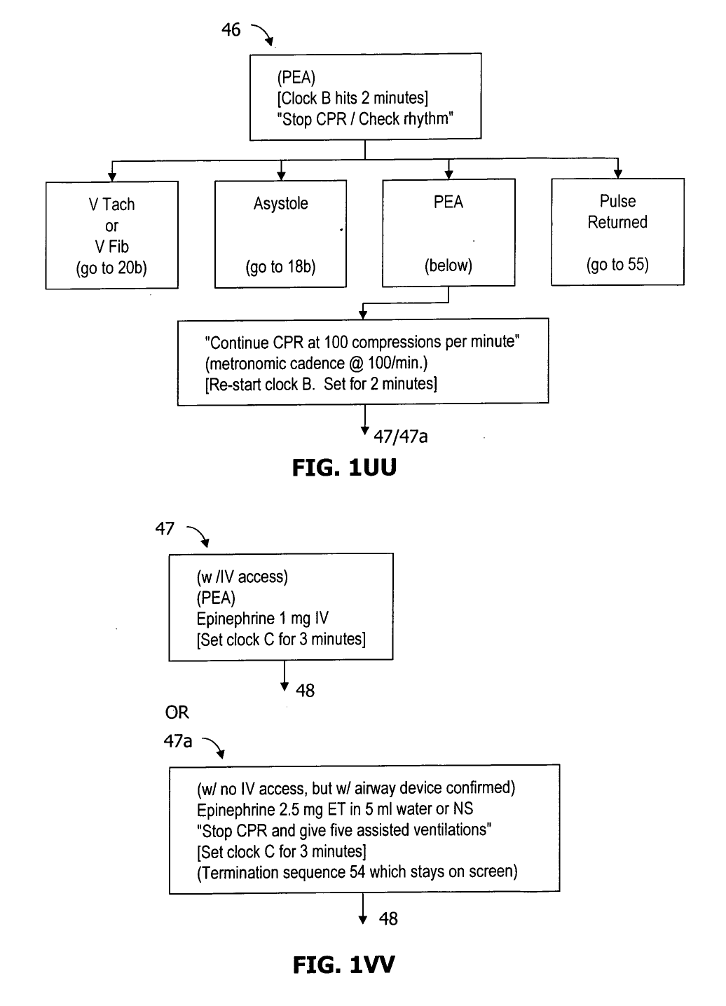

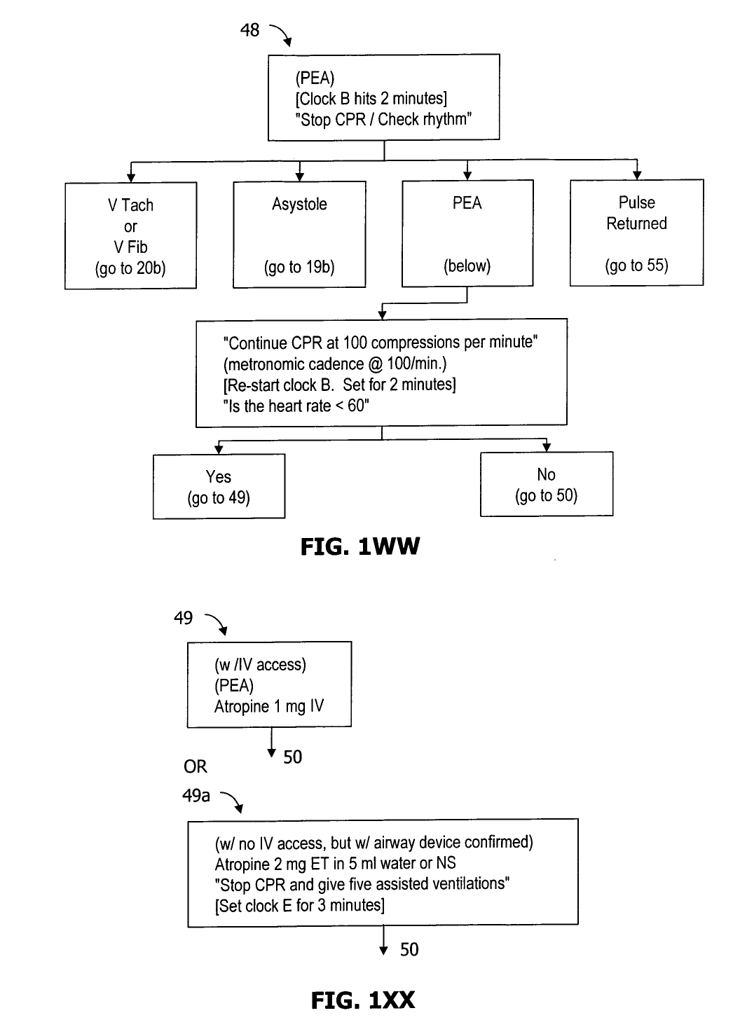

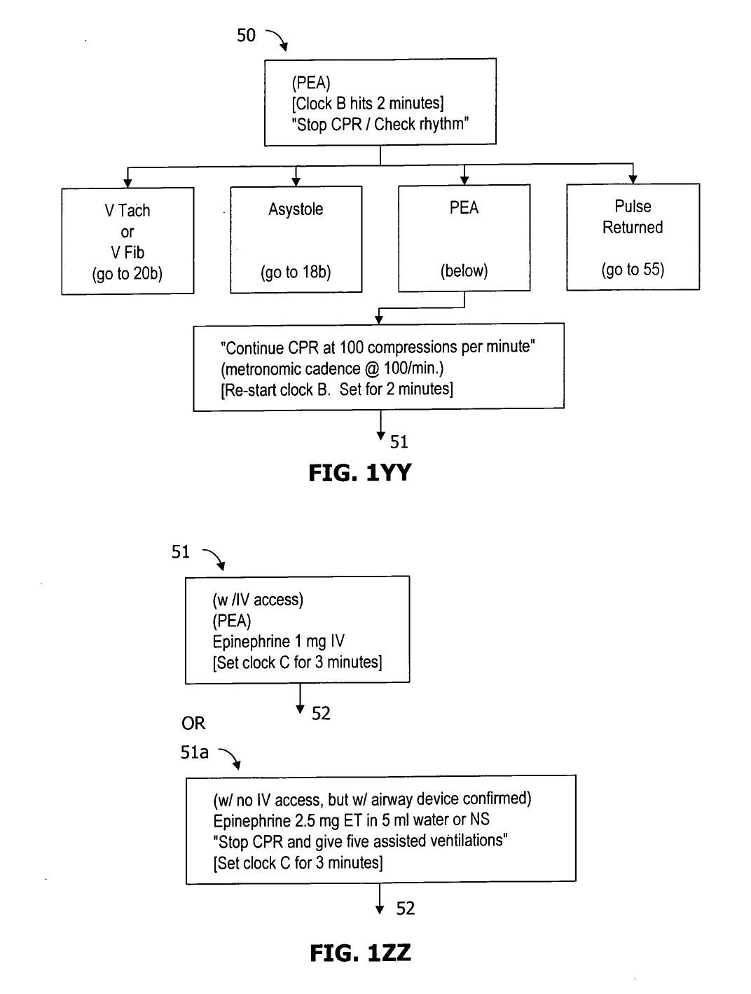

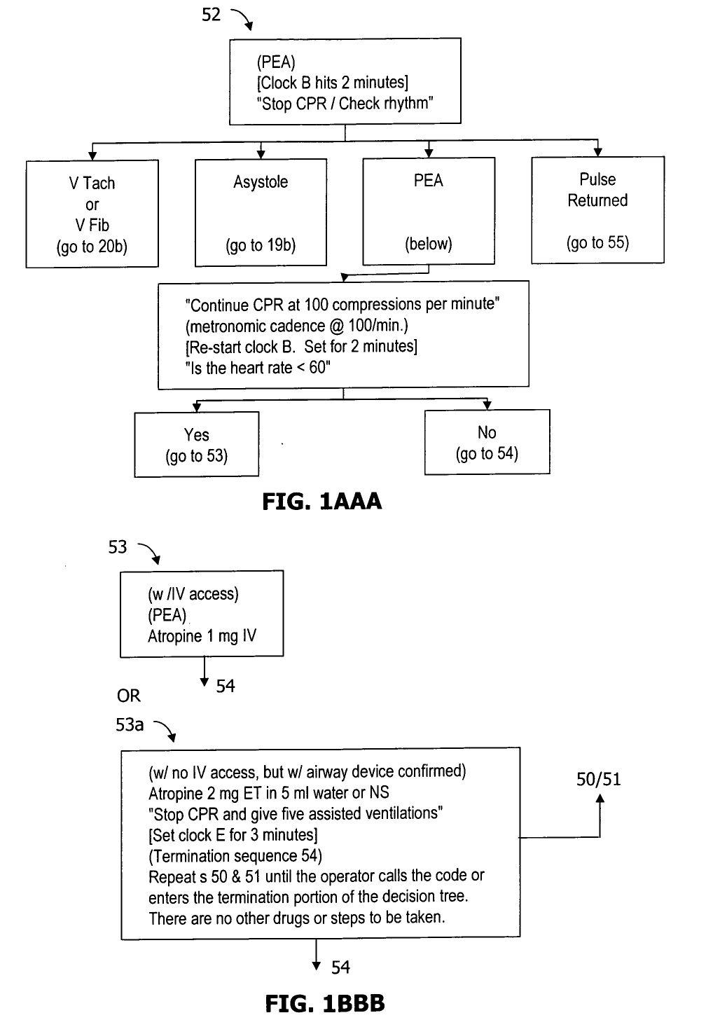

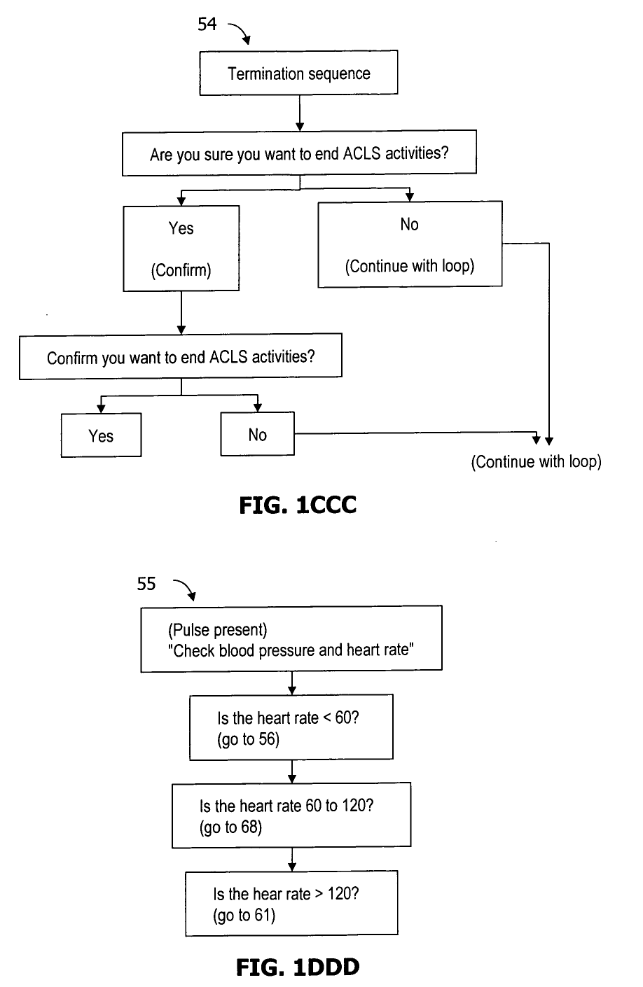

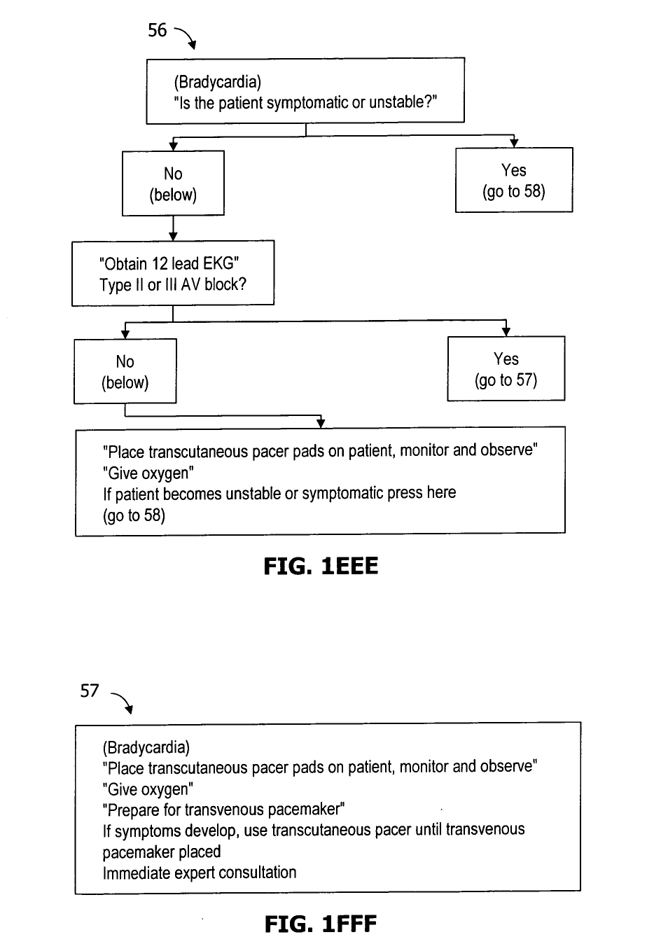

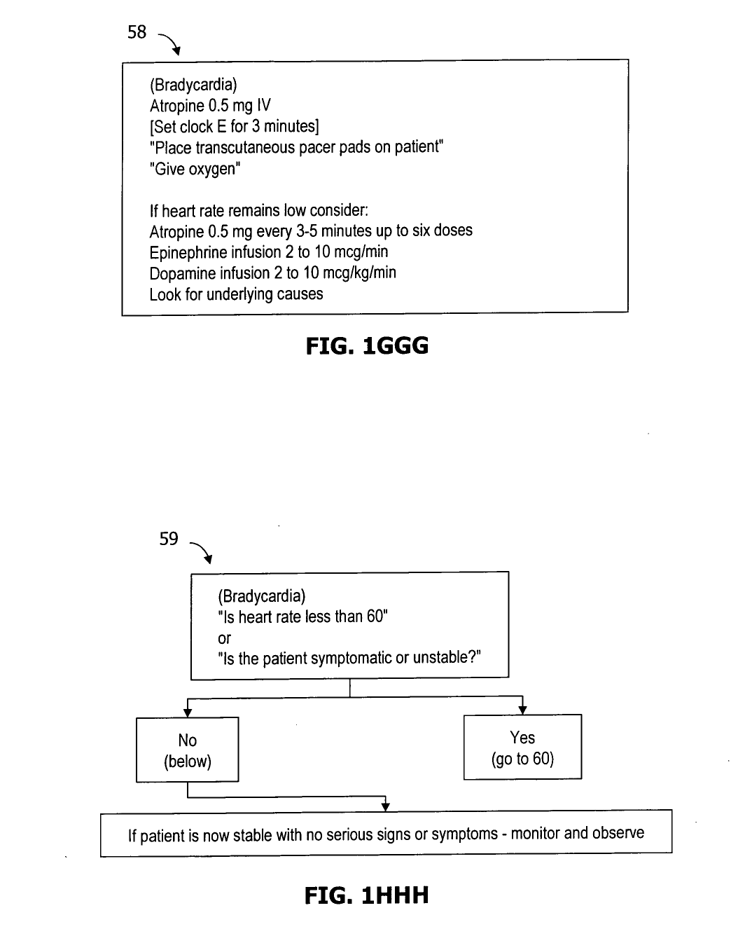

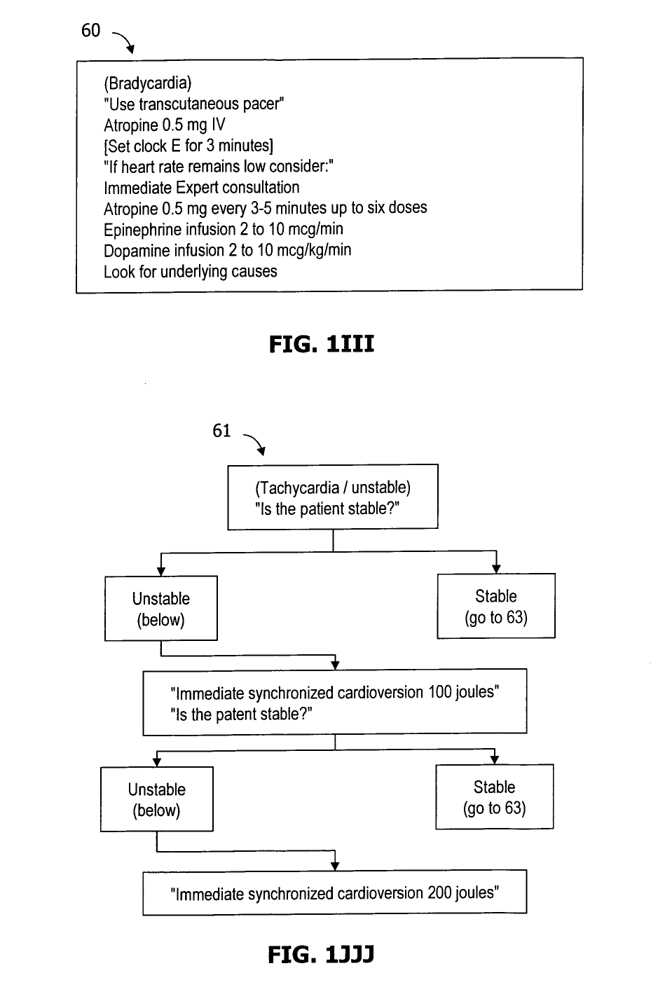

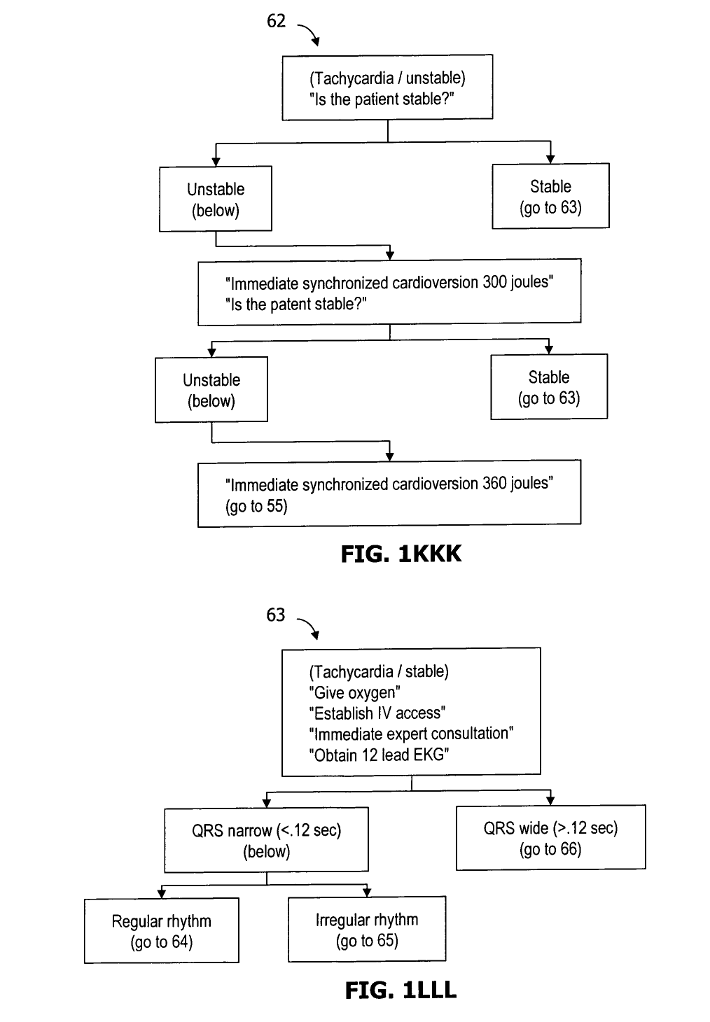

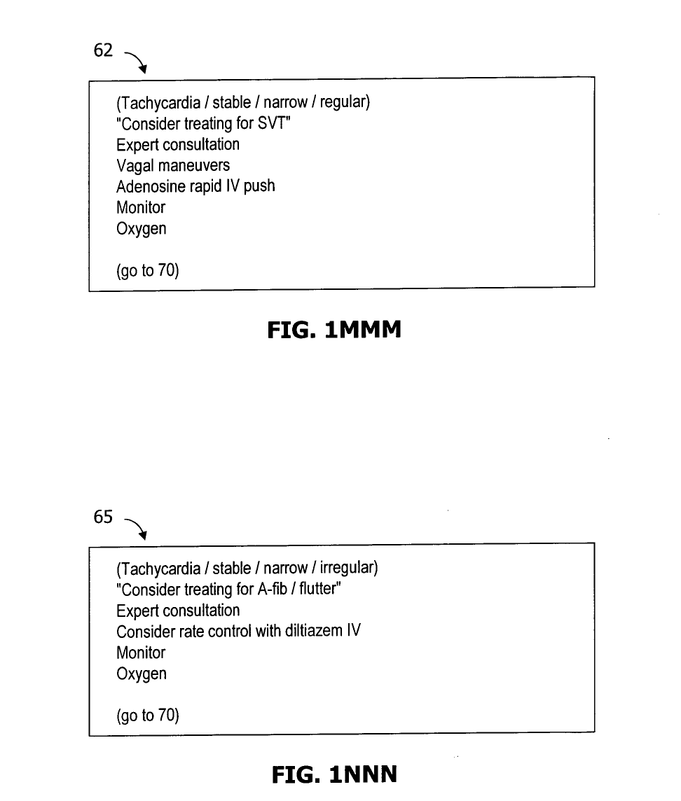

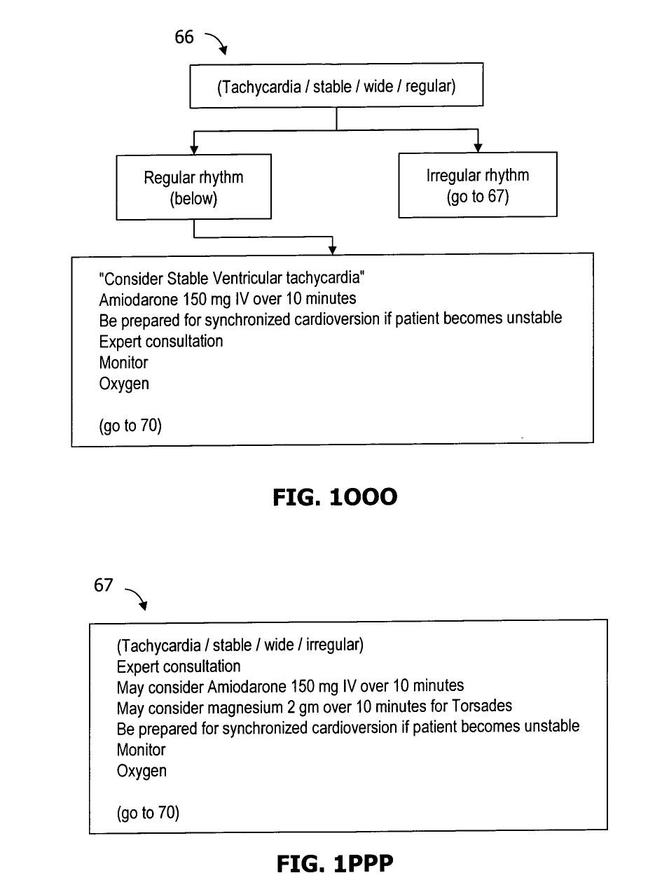

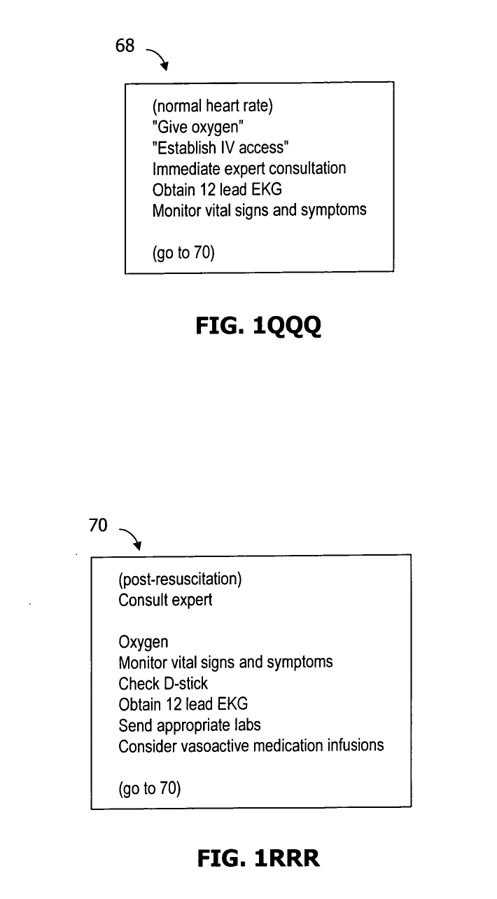

FIGS. 1A-1TTT provide a detailed flow chart of the method that can be implemented in a first embodiment of the apparatus of the present invention, which is used to facilitate resuscitation of adults. The method can modified as described herein so as to facilitate the resuscitation of children. As set forth hereafter, all text in quotations are commands or prompts communicated by the apparatus of the first embodiment of the present invention to the responder, either audibly, visually or both via a sound transducer, touch screen, video display, and the like. Further, as noted above, a plurality of timers are used in the first embodiment of the present invention, which are used to determine when certain actions are to be taken. The first embodiment of the present invention preferably utilizes six (6) timers (hereinafter referred to as clocks) as follows:

External clock A. This clock keeps the elapsed time from the beginning of the ACLS procedure to the end of the ACLS procedure (such elapsed time hereinafter sometimes referred to as the “code” due to its nature as a code blue event) until the user stops the entire event. This clock is displayed on the video display and is visible to the responders and hence is referred to as external.

CPR clock B. This clock is not visible to the responders (and hence is referred to as “internal”) and is on a two minute cycle denoting the length of an individual CPR session. ACLS guidelines are very specific about the importance of chest compressions over medications.

Vasopressor clock C. This is also an internal clock which tracks the timing of this class of medications (Epinephrine or Vasopressin). It cycles every three minutes.

Antiarrythmic clock D. This internal clock cycles every three minutes and tracks the timing of Epinephrine or Amiodarone.

Atropine clock E. This internal clock cycles every three minutes and tracks the timing of atropine.

IV/Airway prompt. This [internal] OR [external] clock cycles every five minutes until affirmed by touch screen or other input acknowledgement.

It is noted that the various clocks can be implemented in a computer using a signal clock source. In addition, the first embodiment of the apparatus of the present invention includes an audible metronomic cadence. Current ACLS protocols emphasize the need for better, faster and uninterrupted CPR. The new rate for chest compressions is 100/min. The audible metronic cadence audibly sets the pace for this rate.

Referring now to FIG. 1A, an initial choice between adult and child is prompted. If adult is chosen, the apparatus begins at step 1. If child is chosen, the apparatus performs the steps for the resuscitation of a child, which are steps modified, as described below, from the adult version. The steps are taken automatically by the apparatus (based on the algorithm) following the responses and/or inputs from a user (or sensor) in response to the prompts of, and communications from, the apparatus.

In step 1 as seen in FIG. 1B, external clock A is started. The apparatus communicates: “Attach monitor and defibrillator” and “Check for a pulse”. If a pulse is present, go to step 55. If a pulse is not present, then choose: Ventricular Tachycardia/Ventricular Fibrillation Algorithm (referred to as “V Tach or V Fib” or VT/VF) (go to step 2), Asystole Algorithm (go to step 18) or Pulseless Electrical Activity Algorithm (PEA) (go to step 36).

In step 2 (VT/VF), as seen in FIG. 1C, the apparatus communicates: “Defibrillate at 200 joules” and “All Clear” (three times) and “Begin CPR at 100 compressions per minute”. Then the metronomic cadence commences at 100/min. Clock B is set for 2 minutes and then started. The apparatus then communicates: “Is IV access established?” If the user answers yes, then step 3 commences. If no, then the question is asked every 3 minutes until answered yes. The apparatus then communicates: “Is airway device in place and confirmed?” If no, the question can be asked every 5 minutes until an affirmative response is obtained. Further, the prompt could remain on the touch screen until the IV and/or an airway is established.

In step 3 (VT/VF) as seen in FIG. 1D, clock B reaches 2 minutes and the apparatus communicates: “Stop CPR/Check rhythm”. Four options are then presented: VT/VF (continue below), Asystole (go to step 18(b)), PEA (go to step 36(b)) and Pulse returned (go to step 55). If the first option is chosen, the apparatus communicates: “Defibrillate at 300 joules” and “All Clear” (three times) and “Continue CPR at 100 compressions per minute”. The metronomic cadence commences at 100 beats per minute. Clock B is set at 2 minutes and restarted. At this point, if the IV is established, the apparatus continues to step 4. If the IV is not established, the apparatus continues to prompt until it is established. Meanwhile, if the responder indicates an IV is not established, but an oral airway is in place, the apparatus commences to step 5(a). If oral airway is not in place, the apparatus continues to prompt until an oral airway is in place. The apparatus may indicate that if neither IV nor oral airway is established no medications can be given.

In step 4 (with IV access) (VT/VF) as seen in FIG. 1E, the apparatus communicates medicine, dosage and method of administration: Epinephrine 1 mg IV or Vasopressin 40 units IV. Clock C is set for 3 minutes. The apparatus can be made to indicate that Vasopressin can only be given once. Hence, if it is given at this juncture it is not an option in the next step. However, if it is not given at this step, it may be offered as an option one more time. The foregoing applies to step 4(a) as well. Alternatively, in step 4(a), without IV access, but with airway device confirmed, the apparatus will communicate: Epinephrine 2.5 mg ET in 5 ml water or NS or Vasopressin 40 units ET in 5 ml water or NS. The apparatus will then communicate: “Stop CPR and give five assisted ventilations”. Clock C is then set for 3 minutes.

In step 5 (VT/VF) as seen in FIG. 1F, the apparatus communicates: “Continue CPR at 100 compressions per minute” and the apparatus will provide a metronomic cadence at 100 beats per minute. Once clock B reaches 2 minutes, the apparatus communicates: “Stop CPR/Check rhythm”. Based on the rhythm, four options are offered for user acceptance: VT/VF (continue below), Asystole (go to step 19(b)), PEA (go to step 38(b)) and Pulse returned (go to step 55). If the first option is chosen, the apparatus communicates: “Defibrillate at 360 joules” and “All Clear” (three times) and “Continue CPR at 100 compressions per minute”. The metronomic cadence commences at 100 beats per minute. Clock B is set at 2 minutes and restarted.

In step 6 (with IV access) (VT/VF) as seen in FIG. 1G, the apparatus communicates medicine, dosage and method of administration: Amiodarone 300 mg IV or Lidocaine 1-1.5 mg/kg IV. Clock D is set for 3 minutes. Alternatively, in step 6(a), without IV access, but with airway device confirmed, the apparatus will communicate: Lidocaine 2-3 mg/kg ET in 5 ml water or NS. The apparatus will then communicate: “Stop CPR and give five assisted ventilations”. Clock D is then set for 3 minutes. The apparatus can note that Amiodarone can not be given through an airway device, thus it is not provided as an option. Once it is indicated that an IV is established, the apparatus will no longer communicate a need to perform step 10(a) during the remainder of the protocols.

In step 7, (VT/VF) as seen in FIG. 1H, clock B reaches 2 minutes and the apparatus communicates: “Stop CPR/Check rhythm”. Four options are then presented: VT/VF (continue below), Asystole (go to step 18(b)), PEA (go to step 36(b)) and Pulse returned (go to step 55). If the first option is chosen, the apparatus communicates: “Defibrillate at 360 joules” and “All Clear” (three times) and “Continue CPR at 100 compressions per minute”. The metronomic cadence commences at 100 beats per minute. Clock B is set at 2 minutes and restarted.

In step 8 (with IV access) (VT/VF) as seen in FIG. 11, the apparatus communicates medicine, dosage and method of administration: Epinephrine 1 mg IV or Vasopressin 40 units IV. Clock C is set for 3 minutes. The apparatus can be made to indicate that Vasopressin can only be given once. However, if it is not given at this step, it may not be offered as an option going forward. The foregoing applies to step 8(a) as well. Alternatively, in step 8(a), without IV access, but with airway device confirmed, the apparatus will communicate: Epinephrine 2.5 mg ET in 5 ml water or NS or Vasopressin 40 units ET in 5 ml water or NS. The apparatus will then communicate: “Stop CPR and give five assisted ventilations”. Clock C is then set for 3 minutes.

In step 9 (VT/VF) as seen in FIG. 1J, clock B reaches 2 minutes and the apparatus communicates: “Stop CPR/Check rhythm”. Four options are then presented: VT/VF (continue below), Asystole (go to step 19(b)), PEA (go to step 38(b)) and Pulse returned (go to step 55). If the first option is chosen, the apparatus communicates: “Defibrillate at 360 joules” and “All Clear” (three times) and “Continue CPR at 100 compressions per minute”. The metronomic cadence commences at 100 beats per minute. Clock B is set at 2 minutes and restarted.

In step 10 (with IV access) (VT/VF) as seen in FIG. 1K, the apparatus communicates medicine, dosage and method of administration: Amiodarone 150 mg IV or Lidocaine 1-1.5 mg/kg IV. Clock D is set for 3 minutes. Alternatively, in step 10(a), without IV access, but with airway device confirmed, the apparatus will communicate: Lidocaine 2-3 mg/kg ET in 5 ml water or NS. The apparatus will then communicate: “Stop CPR and give five assisted ventilations”. Clock D is then set for 3 minutes. The apparatus can note that Amiodarone can not be given through an airway device, thus it is not provided as an option. Once it is indicated that an IV is established, the apparatus will no longer communicate a need to perform step 10(a) during the remainder of the protocols.

In step 11 (VT/VF) as seen in FIG. 1L, clock B reaches 2 minutes and the apparatus communicates: “Stop CPR/Check rhythm”. Four options are then presented: VT/VF (continue below), Asystole (go to step 18(b)), PEA (go to step 36(b)) and Pulse returned (go to step 55). If the first option is chosen, the apparatus communicates: “Defibrillate at 360 joules” and “All Clear” (three times) and “Continue CPR at 100 compressions per minute”. The metronomic cadence commences at 100 beats per minute. Clock B is set at 2 minutes and restarted.

In step 12 (with IV access) (VT/VF) as seen in FIG. 1M, the apparatus communicates medicine, dosage and method of administration: Epinephrine 1 mg IV. Clock C is set for 3 minutes. Alternatively, in step 12(a), without IV access, but with airway device confirmed, the apparatus will communicate: Epinephrine 2.5 mg ET in 5 ml water or NS. The apparatus will then communicate: “Stop CPR and give five assisted ventilations”. Clock C is then set for 3 minutes.

In step 13 (VT/VF) as seen in FIG. 1N, clock B reaches 2 minutes and the apparatus communicates: “Stop CPR/Check rhythm”. Four options are then presented: VT/VF (continue below), Asystole (go to step 19(b)), PEA (go to step 38(b)) and Pulse returned (go to step 55). If the first option is chosen, the apparatus communicates: “Defibrillate at 360 joules” and “All Clear” (three times) and “Continue CPR at 100 compressions per minute”. The metronomic cadence commences at 100 beats per minute. Clock B is set at 2 minutes and restarted.

In step 14 (VT/VF) as seen in FIG. 10, the apparatus communicates: “Has the patient received the maximum dose of 3 mg/kg of Lidocaine?” If the response is no, then the apparatus communicates Lidocaine 1-1.5 mg/kg IV and the clock is not set for this drug. Alternatively, the apparatus continues to step 14(a). If the response is yes, then the apparatus continues to step 15. In step 14(a), without IV access, but with airway device confirmed, the apparatus communicates: Lidocaine 2-3 mg/kg ET in 5 ml water or NS. The apparatus will then communicate: “Stop CPR and give five assisted ventilations”.

In step 15 (reversible causes) as seen in FIG. 1P, the apparatus communicates: “Have you considered the following reversible causes?” The following prompts are then communicated: Hypovolemia, Drug overdose, Hypoxia, Cardiac tamponade, Acidosis, Tension pneumothorax, Hyperkalemia, Pulmonary embolism, Hypokalemia, Coronary thrombosis, Hypothermia. The user is then given the option to save the list for later reference or hide the list. The apparatus may include a prompt button or for this list on the screen at this time so if it needs to be referenced again the user has easy access to it.

In step 16 (VT/VF) as seen in FIG. 1Q, clock B reaches 2 minutes and the apparatus communicates: “Stop CPR/Check rhythm”. Four options are then presented: VT/VF (continue below), Asystole (go to step 18(b)), PEA (go to step 36(b)) and Pulse returned (go to step 55). If the first option is chosen, the apparatus communicates: “Defibrillate at 360 joules” and “All Clear” (three times) and “Continue CPR at 100 compressions per minute”. The metronomic cadence commences at 100 beats per minute. Clock B is set at 2 minutes and restarted.

In step 17 (with IV access) (VT/VF) as seen in FIG. 1R, the apparatus communicates medicine, dosage and method of administration: Epinephrine 1 mg IV. Clock C is set for 3 minutes. Alternatively, in step 17(a), without IV access, but with airway device confirmed, the apparatus will communicate: Epinephrine 2.5 mg ET in 5 ml water or NS. The apparatus will then communicate: “Stop CPR and give five assisted ventilations”. Clock C is then set for 3 minutes. At this point in the method, the apparatus performs a nested loop of steps 16 and 17. There are no new medications until the team is ready to “call the code” and pronounce the patient dead. At that point, the termination sequence stays on screen until step 54.

In step 18 (Asystole), as seen in FIG. 1S, the apparatus communicates: “Confirm asystole in two leads” and “Begin CPR at 100 compressions per minute”. Clock B is then set for 2 minutes and started. The apparatus then prompts: “Is IV access established?” in accordance with step 3. If so, the apparatus continues to step 19. If not, the prompt is continued and asked again every five minutes until an affirmative response is provided. Meanwhile, the apparatus communicates: “Is airway device in place and confirmed?” The prompt is continued and asked again every five minutes until an affirmative response is provided. Further, the prompt could remain on the touch screen until the IV and/or an airway is established. Alternatively, in step 18(a) (Asystole), the apparatus communicates: “Confirm asystole in two leads” and “Begin CPR at 100 compressions per minute”. The metronomic cadence commences at 100 beats per minute. Clock B is set at 2 minutes and restarted. Information about the IV and airway from this step is stored in computer memory.

In step 19 (with IV access) (Asystole) as seen in FIG. 1T, the apparatus communicates: “Consider transcutaneous pacing”. The apparatus communicates medicine, dosage and method of administration: Epinephrine 1 mg IV or Vasopressin 40 units IV. Clock C is set for 3 minutes. The apparatus can be made to indicate that Vasopressin can only be given once. Hence, if it is given at this juncture it is not an option for the next step. However, if it is not given at this step, it may be offered as an option one more time. Alternatively, in step 19(a), without IV access, but with airway device confirmed, the apparatus will communicate: Epinephrine 2.5 mg ET in 5 ml water or NS or Vasopressin 40 units ET in 5 ml water or NS. The apparatus will then communicate: “Stop CPR and give five assisted ventilations”. Clock C is then set for 3 minutes. And the apparatus continues to step 20. At step 19(b) (Asystole), as seen in FIG. 1U, the apparatus communicates: “Confirm asystole in two leads” and “Begin CPR at 100 compressions per minute”. Clock B is then set for 2 minutes and started.

In step 20 (Asystole) as seen in FIG. 1U the apparatus communicates: “Consider transcutaneous pacing”. Clock B reaches 2 minutes and the apparatus communicates: “Stop CPR/Check rhythm”. Four options are then presented: VT/VF (continue to step 20(b)), Asystole (continue below), PEA (go to step 38(b)) and Pulse returned (go to step 55). The apparatus communicates: “Continue CPR at 100 compressions per minute”. The metronomic cadence commences at 100 beats per minute. Clock B is set at 2 minutes and restarted.

In step 20(b) (VT/VF) the apparatus communicates: “Defibrillate at 200 joules” and “All Clear” (three times) and “Continue CPR at 100 compressions per minute”. The metronomic cadence commences at 100 beats per minute. Clock B is set at 2 minutes and restarted. The apparatus then continues to step 4.

In step 21 (with IV access) (Asystole) as seen in FIG. 1V the apparatus communicates medicine, dosage and method of administration: Atropine 1 mg IV. Alternatively, in step 21(a), without IV access, but with airway device confirmed, the apparatus will communicate: Atropine 2 mg ET in 5 ml water or NS. The apparatus will then communicate: “Stop CPR and give five assisted ventilations”. Clock E is then set for 3 minutes. At this point in the method, the apparatus can remind the responder that that Atropine can only be given a maximum of four times total, and such fact is coded into the application software.

In step 22 (reversible causes) as seen in FIG. 1W the apparatus communicates: “Have you considered the following reversible causes?” The following prompts are then communicated: Hypovolemia, Drug overdose, Hypoxia, Cardiac tamponade, Acidosis, Tension pneumothorax, Hyperkalemia, Pulmonary embolism, Hypokalemia, Coronary thrombosis, Hypothermia. The user is then given the option to save the list for later reference or hide the list. The apparatus may include a prompt button or for this list on the screen at this time so if it needs to be referenced again the user has easy access to it.

In step 23 (Asystole) as seen in FIG. 1X clock B reaches 2 minutes and the apparatus communicates: “Stop CPR/Check rhythm”. Four options are then presented: VT/VF (continue to step 20(b)), Asystole (continue below), PEA (go to step 36(b)) and Pulse returned (go to step 55). The apparatus communicates: “Continue CPR at 100 compressions per minute”. The metronomic cadence commences at 100 beats per minute. Clock B is set at 2 minutes and restarted.

In step 24 (with IV access) (Asystole) as seen in FIG. 1Y, the apparatus communicates medicine, dosage and method of administration: Epinephrine 1 mg IV or Vasopressin 40 units IV. Clock C is set for 3 minutes. The apparatus can be made to indicate that Vasopressin can only be given once. Hence, if it is given at this juncture it is not an option for the next step. However, if it is not given at this step, it may be offered as an option one more time. Alternatively, in step 24(a), without IV access, but with airway device confirmed, the apparatus will communicate: Epinephrine 2.5 mg ET in 5 ml water or NS or Vasopressin 40 units ET in 5 ml water or NS. The apparatus will then communicate: “Stop CPR and give five assisted ventilations”. Clock C is then set for 3 minutes.

In step 25 (Asystole) as seen in FIG. 1Z, the apparatus communicates: “Stop CPR/Check rhythm”. Four options are then presented: VT/VF (continue to step 20(b)), Asystole (continue below), PEA (go to step 38(b)) and Pulse returned (go to step 55). The apparatus communicates: “Continue CPR at 100 compressions per minute”. The metronomic cadence commences at 100 beats per minute. Clock B is set at 2 minutes and restarted.

In step 26 (with IV access) (Asystole) as seen in FIG. 1 AA, the apparatus communicates medicine, dosage and method of administration: Atropine 1 mg IV. Alternatively, in step 26(a), without IV access, but with airway device confirmed, the apparatus will communicate: Atropine 2 mg ET in 5 ml water or NS. The apparatus will then communicate: “Stop CPR and give five assisted ventilations”. Clock E is then set for 3 minutes. It is flagged internally by the apparatus that this is the second of four total doses of Atropine that can be given. Such fact can also be communicated to the responder as a reminder.

In step 27 (reversible causes) as seen in FIG. 1BB, the apparatus communicates: “Have you considered the following reversible causes?” The following prompts are then communicated: Hypovolemia, Drug overdose, Hypoxia, Cardiac tamponade, Acidosis, Tension pneumothorax, Hyperkalemia, Pulmonary embolism, Hypokalemia, Coronary thrombosis, Hypothermia. The user is then given the option to save the list for later reference or hide the list. The apparatus may include a prompt button or for this list on the screen at this time so if it needs to be referenced again the user has easy access to it.

In step 28 (Asystole) as seen in FIG. 1CC, clock B reaches 2 minutes and the apparatus communicates: “Stop CPR/Check rhythm”. Four options are then presented: VT/VF (continue to step 20(b)), Asystole (continue below), PEA (go to step 36(b)) and Pulse returned (go to step 55). The apparatus communicates: “Continue CPR at 100 compressions per minute”. The metronomic cadence commences at 100 beats per minute. Clock B is set at 2 minutes and restarted.

In step 29 (with IV access) (Asystole) as seen in FIG. 1 DD, the apparatus communicates medicine, dosage and method of administration: Epinephrine 1 mg IV. Clock C is set for 3 minutes. Alternatively, in step 29(a), without IV access, but with airway device confirmed, the apparatus will communicate: Epinephrine 2.5 mg ET in 5 ml water or NS. The apparatus will then communicate: “Stop CPR and give five assisted ventilations”. Clock C is then set for 3 minutes. At this point, a termination sequence remains on the video display until step 54.

In step 30 (Asystole) as seen in FIG. 1EE, the apparatus communicates: “Stop CPR/Check rhythm”. Four options are then presented: VT/VF (continue to step 20(b)), Asystole (continue below), PEA (go to step 38(b)) and Pulse returned (go to step 55). The apparatus communicates: “Continue CPR at 100 compressions per minute”. The metronomic cadence commences at 100 beats per minute. Clock B is set at 2 minutes and restarted.

In step 31 (with IV access) (Asystole) as seen in FIG. 1FF, the apparatus communicates medicine, dosage and method of administration: Atropine 1 mg IV. Alternatively, in step 31(a), without IV access, but with airway device confirmed, the apparatus will communicate: Atropine 2 mg ET in 5 ml water or NS. The apparatus will then communicate: “Stop CPR and give five assisted ventilations”. Clock E is then set for 3 minutes. It is flagged internally by the apparatus that this is the third of four total doses of Atropine that can be given. Such fact can also be communicated to the responder as a reminder.

In step 32 (Asystole) as seen in FIG. 1GG, clock B reaches 2 minutes and the apparatus communicates: “Stop CPR/Check rhythm”. Four options are then presented: VT/VF (continue to step 20(b)), Asystole (continue below), PEA (go to step 36(b)) and Pulse returned (go to step 55). The apparatus communicates: “Continue CPR at 100 compressions per minute”. The metronomic cadence commences at 100 beats per minute. Clock B is set at 2 minutes and restarted.

In step 33 (with IV access) (Asystole) as seen in FIG. 1HH, the apparatus communicates medicine, dosage and method of administration: Epinephrine 1 mg IV. Clock C is set for 3 minutes. Alternatively, in step 33(a), without IV access, but with airway device confirmed, the apparatus will communicate: Epinephrine 2.5 mg ET in 5 ml water or NS. The apparatus will then communicate: “Stop CPR and give five assisted ventilations”. Clock C is then set for 3 minutes.

In step 34 (Asystole) as seen in FIG. 1II, clock B reaches 2 minutes and the apparatus communicates: “Stop CPR/Check rhythm”. Four options are then presented: VT/VF (continue to step 20(b)), Asystole (continue below), PEA (go to step 38(b)) and Pulse returned (go to step 55). The apparatus communicates: “Continue CPR at 100 compressions per minute”. The metronomic cadence commences at 100 beats per minute. Clock B is set at 2 minutes and restarted.

In step 35 (with IV access) (Asystole) as seen in FIG. 1JJ, the apparatus communicates medicine, dosage and method of administration: Atropine 1 mg IV. Alternatively, in step 35(a), without IV access, but with airway device confirmed, the apparatus will communicate: Atropine 2 mg ET in 5 ml water or NS. The apparatus will then communicate: “Stop CPR and give five assisted ventilations”. Clock E is then set for 3 minutes. It is flagged internally by the apparatus that this is the fourth of four total doses of Atropine that can be given. Such fact can also be communicated to the responder as a reminder. At this point, a termination sequence remains on the video display from step 54. Steps 32 and 33 are looped until the responder calls the code or the termination portion of the method is invoked. At this point, there are no further drugs to be administered of actions to be taken.

In step 36 (PEA) as seen in FIG. 1KK, the apparatus communicates: “Begin CPR at 100 compressions per minute”. Then the metronomic cadence commences at 100 beats per minute. Clock B is set for 2 minutes and then started. The apparatus then communicates: “Is IV access established?” If the user answers yes, then the method returns to step 3. If no, then the question is asked every 5 minutes until answered yes. The apparatus then communicates: “Is airway device in place and confirmed?” If no, the question can be asked every 5 minutes until an affirmative response is obtained. Further, the prompt could remain on the touch screen until the IV and/or an airway is established. If yes, the method continues to step 37. In step 36(b) (PEA) as seen in FIG. 1NN, the apparatus communicates: “Begin CPR at 100 compressions per minute”. Then the metronomic cadence commences at 100 beats per minute. Clock B is set for 2 minutes and then started.

In step 37 (with IV access) (PEA) as seen in FIG. ILL, the apparatus communicates medicine, dosage and method of administration: Epinephrine 1 mg IV or Vasopressin 40 units IV. Clock C is set for 3 minutes. The apparatus can be made to indicate that Vasopressin can only be given once. Hence, if it is given at this juncture it is not an option in the next step. However, if it is not given at this step, it may be offered as an option one more time. Alternatively, in step 37(a), without IV access, but with airway device confirmed, the apparatus will communicate: Epinephrine 2.5 mg ET in 5 ml water or NS or Vasopressin 40 units ET in 5 ml water or NS. The apparatus will then communicate: “Stop CPR and give five assisted ventilations”. Clock C is then set for 3 minutes.

In step 38 (PEA) as seen in FIG. 1MM, once clock B reaches 2 minutes, the apparatus communicates: “Stop CPR/Check rhythm”. Based on the rhythm, four options are offered for user acceptance: VT/VF (go to step 20(b)), Asystole (go to step 19(b)), PEA (continue below) and Pulse returned (go to step 55). The apparatus communicates: “Continue CPR at 100 compressions per minute”. The metronomic cadence commences at 100 beats per minute. Clock B is set at 2 minutes and restarted. The apparatus communicates: “Is the heart rate less than 60” If the user selects yes, the method continues to step 39. If the user selects no, the method continues to step 40.

In step 39 (with IV access) (PEA) as seen in FIG. 1NN, the apparatus communicates medicine, dosage and method of administration: Atropine 1 mg IV. Alternatively, in step 39(a), without IV access, but with airway device confirmed, the apparatus will communicate: Atropine 2 mg ET in 5 ml water or NS. The apparatus will then communicate: “Stop CPR and give five assisted ventilations”. Clock E is then set for 3 minutes. It is flagged internally by the apparatus that this is the first of four total doses of Atropine that can be given. Such fact can also be communicated to the responder as a reminder.

In step 40 (reversible causes) as seen in FIG. 1OO, the apparatus communicates: “Have you considered the following reversible causes?” The following prompts are then communicated: Hypovolemia, Drug overdose, Hypoxia, Cardiac tamponade, Acidosis, Tension pneumothorax, Hyperkalemia, Pulmonary embolism, Hypokalemia, Coronary thrombosis, Hypothermia. The user is then given the option to save the list for later reference or hide the list. The apparatus may include a prompt button or for this list on the screen at this time so if it needs to be referenced again the user has easy access to it.

In step 41 (PEA) as seen in FIG. 1PP, clock B reaches 2 minutes and the apparatus communicates: “Stop CPR/Check rhythm”. Four options are then presented: VT/VF (continue to step 20(b)), Asystole (go to step 18(b)), PEA (continue below) and Pulse returned (go to step 55). The apparatus communicates: “Continue CPR at 100 compressions per minute”. The metronomic cadence commences at 100 beats per minute. Clock B is set at 2 minutes and restarted.

In step 42 (with IV access) (PEA) as seen in FIG. 1QQ, the apparatus communicates medicine, dosage and method of administration: Epinephrine 1 mg IV or Vasopressin 40 units IV. Clock C is set for 3 minutes. The apparatus can be made to indicate that Vasopressin can only be given once. Hence, if it is given at this juncture it is not an option for the next step. However, if it is not given at this step, it may be offered as an option one more time. Alternatively, in step 42(a), without IV access, but with airway device confirmed, the apparatus will communicate: Epinephrine 2.5 mg ET in 5 ml water or NS or Vasopressin 40 units ET in 5 ml water or NS. The apparatus will then communicate: “Stop CPR and give five assisted ventilations”. Clock C is then set for 3 minutes.

In step 43 (PEA) as seen in FIG. 1RR, clock B reaches 2 minutes and the apparatus communicates: “Stop CPR/Check rhythm”. Four options are then presented: VT/VF (continue to step 20(b)), Asystole (go to step 19(b)), PEA (continue below) and Pulse returned (go to step 55). The apparatus communicates: “Continue CPR at 100 compressions per minute”. The metronomic cadence commences at 100 beats per minute. Clock B is set at 2 minutes and restarted. The apparatus communicates: “Is the heart rate less than 60” If the user selects yes, the method continues to step 44. If the user selects no, the method continues to step 45.

In step 44 (with IV access) (PEA) as seen in FIG. 1SS, the apparatus communicates medicine, dosage and method of administration: Atropine 1 mg IV. Alternatively, in step 44(a), without IV access, but with airway device confirmed, the apparatus will communicate: Atropine 2 mg ET in 5 ml water or NS. The apparatus will then communicate: “Stop CPR and give five assisted ventilations”. Clock E is then set for 3 minutes. It is flagged internally by the apparatus that this is the second of four total doses of Atropine that can be given. Such fact can also be communicated to the responder as a reminder.

In step 45 (reversible causes) as seen in FIG. 1TT, the apparatus communicates: “Have you considered the following reversible causes?” The following prompts are then communicated: Hypovolemia, Drug overdose, Hypoxia, Cardiac tamponade, Acidosis, Tension pneumothorax, Hyperkalemia, Pulmonary embolism, Hypokalemia, Coronary thrombosis, Hypothermia. The user is then given the option to save the list for later reference or hide the list. The apparatus may include a prompt button or for this list on the screen at this time so if it needs to be referenced again the user has easy access to it.

In step 46 (PEA) as seen in FIG. 1UU, clock B reaches 2 minutes and the apparatus communicates: “Stop CPR/Check rhythm”. Four options are then presented: VT/VF (continue to step 20(b)), Asystole (go to step 18(b)), PEA (continue below) and Pulse returned (go to step 55). The apparatus communicates: “Continue CPR at 100 compressions per minute”. The metronomic cadence commences at 100 beats per minute. Clock B is set at 2 minutes and restarted.

In step 47 (with IV access) (PEA) as seen in FIG. 1VV, the apparatus communicates medicine, dosage and method of administration: Epinephrine 1 mg IV. Clock C is set for 3 minutes. Alternatively, in step 47(a), without IV access, but with airway device confirmed, the apparatus will communicate: Epinephrine 2.5 mg ET in 5 ml water or NS. The apparatus will then communicate: “Stop CPR and give five assisted ventilations”. Clock C is then set for 3 minutes. At this point, a termination sequence remains on the video display until step 54.

In step 48 (PEA) as seen in FIG. 1WW, the apparatus communicates: “Stop CPR/Check rhythm”. Four options are then presented: VT/VF (continue to step 20(b)), Asystole (go to step 19(b)), PEA (continue below) and Pulse returned (go to step 55). The apparatus communicates: “Continue CPR at 100 compressions per minute”. The metronomic cadence commences at 100 beats per minute. Clock B is set at 2 minutes and restarted. The apparatus communicates: “Is the heart rate less than 60” If the user selects yes, the method continues to step 49. If the user selects no, the method continues to step 50.

In step 49 (with IV access) (PEA) as seen in FIG. 1XX, the apparatus communicates medicine, dosage and method of administration: Atropine 1 mg IV. Alternatively, in step 49(a), without IV access, but with airway device confirmed, the apparatus will communicate: Atropine 2 mg ET in 5 ml water or NS. The apparatus will then communicate: “Stop CPR and give five assisted ventilations”. Clock E is then set for 3 minutes. It is flagged internally by the apparatus that this is the third of four total doses of Atropine that can be given. Such fact can also be communicated to the responder as a reminder.

In step 50 (PEA) as seen in FIG. 1YY, clock B reaches 2 minutes and the apparatus communicates: “Stop CPR/Check rhythm”. Four options are then presented: VT/VF (continue to step 20(b)), Asystole (go to step 18(b)), PEA (continue below) and Pulse returned (go to step 55). The apparatus communicates: “Continue CPR at 100 compressions per minute”. The metronomic cadence commences at 100 beats per minute. Clock B is set at 2 minutes and restarted.

In step 51 (with IV access) (PEA) as seen in FIG. 1ZZ, the apparatus communicates medicine, dosage and method of administration: Epinephrine 1 mg IV. Clock C is set for 3 minutes. Alternatively, in step 51(a), without IV access, but with airway device confirmed, the apparatus will communicate: Epinephrine 2.5 mg ET in 5 ml water or NS. The apparatus will then communicate: “Stop CPR and give five assisted ventilations”. Clock C is then set for 3 minutes.

In step 52 (PEA) as seen in FIG. 1AAA, clock B reaches 2 minutes and the apparatus communicates: “Stop CPR/Check rhythm”. Four options are then presented: VT/VF (continue to step 20(b)), Asystole (go to step 19(b)), PEA (continue below) and Pulse returned (go to step 55). The apparatus communicates: “Continue CPR at 100 compressions per minute”. The metronomic cadence commences at 100 beats per minute. Clock B is set at 2 minutes and restarted. The apparatus communicates: “Is the heart rate less than 60” If the user selects yes, the method continues to step 53. If the user selects no, the method continues to step 54.

In step 53 (with IV access) (PEA) as seen in FIG. 1BBB, the apparatus communicates medicine, dosage and method of administration: Atropine 1 mg IV. Alternatively, in step 53(a), without IV access, but with airway device confirmed, the apparatus will communicate: Atropine 2 mg ET in 5 ml water or NS. The apparatus will then communicate: “Stop CPR and give five assisted ventilations”. Clock E is then set for 3 minutes. It is flagged internally by the apparatus that this is the fourth of four total doses of Atropine that can be given. Such fact can also be communicated to the responder as a reminder. At this point, a termination sequence remains on the video display from step 54. Steps 50 and 51 are looped until the responder calls the code or the termination portion of the method is invoked. At this point, there are no further drugs to be administered of actions to be taken.

In step 54 as seen in FIG. 1CCC, the termination sequence is invoked. The apparatus communicates: “Are you sure you want to end ACLS activities?” If yes, then the apparatus communicates: “Confirm you want to end ACLS activities?” If no, the loop continues. At this point, the user is given an option to print a summary of events with the timing of drugs given relative to the external clocks and the internal elapsed time clock. In one aspect of the invention, a sheet of paper having an adhesive back that can be inserted into the apparatus, printed on and then ejected from the apparatus.

In step 55 (Pulse present) as seen in FIG. 1DDD, the apparatus communicates: “Check blood pressure and heart rate”. Three options are then presented: Is the heart rate less than 60? (go to step 56); Is the heart rate 60 to 120? (go to step 68) Is the heart rate greater than 120? (go to step 61).

In step 56 (Bradycardia) as seen in FIG. IEEE, the apparatus communicates: “Is the patient symptomatic or unstable?” Two options are presented for selection. If no, then continue below. If yes, then (go to step 58). The apparatus communicates: “Obtain 12 lead EKG”. Then two options are presented for selection. Type II or III AV block? If no, then continue below. If yes, then go to step 57. The apparatus communicates: “Place transcutaneous pacer pads on patient, monitor and observe,” and “Give oxygen”. An option is then presented: If patient becomes unstable or symptomatic press here. If the option is selected, the method goes to step 58.

In step 57 (Bradycardia) as seen in FIG. 1FFF, the apparatus communicates “Place transcutaneous pacer pads on patient, monitor and observe,” and “Give oxygen” and “Prepare for transvenous pacemaker”. The responder may then be reminded and prompted: If symptoms develop, use transcutaneous pacer until transvenous pacemaker placed and Immediate expert consultation.

In step 58, (Bradycardia) as seen in FIG. 1GGG, the apparatus communicates medicine, dosage and method of administration: Atropine 0.5 mg W. Clock E is set for 3 minutes. The apparatus communicates: “Place transcutaneous pacer pads on patient” and “Give oxygen” and “If heart rate remains low consider:” and the following are displayed on the video display: Atropine 0.5 mg every 3-5 minutes up to six doses; Epinephrine infusion 2 to 10 mcg/min; Dopamine infusion 2 to 10 mcg/kg/min; and Look for underlying causes.

In step 59 (Bradycardia) as seen in FIG. 1HHH, the apparatus communicates: “Is the heart rate less than 60” or “Is the patient symptomatic or unstable?” Two options are presented for selection. If no, then continue below. If yes is selected (go to step 60). The apparatus will then prompt: If patient is now stable with no serious signs or symptoms—monitor and observe.

In step 60 (Bradycardia) as seen in FIG. 1III, the apparatus communicates “Use transcutaneous pacer”. The apparatus communicates medicine, dosage and method of administration: Atropine 0.5 mg IV. Clock E is set for 3 minutes. The apparatus communicates: “If heart rate remains low consider:” and the following are displayed on the video display: Immediate Expert consultation; Atropine 0.5 mg every 3-5 minutes up to six doses; Epinephrine infusion 2 to 10 mcg/min; Dopamine infusion 2 to 10 mcg/kg/min; and Look for underlying causes.

In step 61 (Bradycardia/unstable) as seen in FIG. 1JJJ, the apparatus communicates: “Is the patient stable?” Two options are presented for selection. Unstable (continue below) and stable (go to step 63). The apparatus communicates: “Immediate synchronized cardioversion 100 joules”. The apparatus communicates: “Is the patient stable?” If unstable, continue below. If stable is selected, go to step 63. The apparatus communicates: “Immediate synchronized cardioversion 200 joules”.

In step 62 (Bradycardia/unstable) as seen in FIG. 1KKK, the apparatus communicates: “Is the patient stable?” Two options are presented for selection. Unstable (continue below) and stable (go to step 63). The apparatus communicates: “Immediate synchronized cardioversion 300 joules”. The apparatus communicates: “Is the patient stable?” If unstable, continue below. If stable is selected, go to step 63. The apparatus communicates: “Immediate synchronized cardioversion 360 joules”. The method then goes to step 55.

In step 63 (Tachycardia/stable) as seen in FIG. 1LLL, the apparatus communicates: “Give oxygen” and “Establish IV access” and “Immediate expert consultation” and “Obtain 12 lead EKG”. Four options are then presented for selection: QRS narrow (<0.12 sec) (continue below); QRS wide (>0.12 sec) (go to step 66); Regular rhythm (go to step 64) and Irregular rhythm (go to step 65).

In step 64 (Tachycardia/stable/narrow/regular) as seen in FIG. 1MMM, the apparatus communicates: “Consider treating for SVT” and the following reminders are provided: Expert consultation; Vagal maneuvers; Adenosine rapid IV push; Monitor; Oxygen. The apparatus then continues to step 70.

In step 65 (Tachycardia/stable/narrow/irregular) as seen in FIG. 1NNN, the apparatus communicates: “Consider treating for A-fib/flutter” and the following reminders are provided: Expert consultation; Consider rate control with diltiazem IV; Monitor; Oxygen. The apparatus then continues to step 70.

In step 66 (Tachycardia/stable/narrow/regular) as seen in FIG. 1OOO, the apparatus provides two options: Regular rhythm (continue below) and Irregular rhythm (go to step 67). The apparatus then communicates: “Consider Stable Ventricular tachycardia” and the following reminders are provided: Amiodarone 150 mg IV over 10 minutes; Be prepared for synchronized cardioversion if patient becomes unstable; Expert consultation; Monitor; Oxygen. The apparatus then continues to step 70.

In step 67 (Tachycardia/stable/wide/irregular) as seen in FIG. 1PPP, the apparatus provides the following reminders: Expert consultation; May consider Amiodarone 150 mg IV over 10 minutes; May consider magnesium 2 gm over 10 minutes for Torsades; Be prepared for synchronized cardioversion if patient becomes unstable; Monitor; Oxygen. The apparatus then continues to step 70.

In step 68 (normal heart rate) as seen in FIG. 1QQQ, the apparatus communicates: “Give oxygen” and “Establish IV access”. The apparatus provides the following reminders: Immediate expert consultation; Obtain 12 lead EKG; and Monitor vital signs and symptoms. The apparatus then continues to step 70.

In step 70 (post-resuscitation), the apparatus provides the following reminders: Consult expert; Oxygen; Monitor vital signs and symptoms; Check D-stick; Obtain 12 lead EKG; Send appropriate labs; Consider vasoactive medication infusions.

The steps taken by the apparatus if the patient selection is a child, are similar to those taken if the patient selected is an adult, with such modifications are necessary due to the physiological differences between adults and children. Such modifications are as follows. In pediatric step 1, external clock A is started. The apparatus communicates: “Check for a pulse” and “Attach monitor and defibrillator”. If a pulse is present, go to pediatric step 31. If a pulse is not present, then “Begin CPR at 100 compressions per minute”. Then the metronomic cadence commences at 100/min. The apparatus communicates: “Check rhythm”. The two choices are offered: VT/VF (go to pediatric step 2), Asystole/PEA Algorithm (go to pediatric step 18).

In pediatric step 2 (VT/VF), the apparatus communicates: “Defibrillate at 2 joules per kilogram” and “All Clear” (three times) and “Continue CPR at 100 compressions per minute”. Then the metronomic cadence commences at 100/min. Clock B is set for 2 minutes and then started. The apparatus then communicates: “Is IV or IO access established?” If the user answers yes, then pediatric step 3 commences. If no, then the question is asked every 3 minutes until answered yes. The apparatus then communicates: “Is airway device in place and confirmed?” If no, the question can be asked every 5 minutes until an affirmative response is obtained. Further, the prompt could remain on the touch screen until the IV and/or an airway is established.

In pediatric step 3 (VT/VF) clock B reaches 2 minutes and the apparatus communicates: “Stop CPR/Check rhythm”. Three options are then presented: VT/VF (continue below), Asystole (go to pediatric step 18), Organized Rhythm (go to pediatric step 28(b)). If the first option is chosen, the apparatus communicates: “Defibrillate at 4 joules per kilogram” and “All Clear” (three times) and “Continue CPR at 100 compressions per minute”. The metronomic cadence commences at 100 beats per minute. Clock B is set at 2 minutes and restarted. At this point, if IV/IO is established, the apparatus continues to pediatric step 4. If the IV/IO are not established, the apparatus continues to prompt until it is established. Meanwhile, if the responder indicates IV/IO are not established, but an oral airway is in place, the apparatus commences to pediatric step 5(a). If an oral airway is not in place, the apparatus continues to prompt until an oral airway is in place. The apparatus may indicate that if neither device is established no medications can be given.

In pediatric step 4 (with IV or IO access) (VT/VF) the apparatus communicates medicine, dosage and method of administration: Epinephrine 0.01 mg IV/IO (1:10,000; 0.1 ml/kg). Clock C is set for 3 minutes. Alternatively, in pediatric step 4(a), without IV/IO access, but with airway device confirmed, the apparatus will communicate: Epinephrine 0.1 mg/kg ET in 5 ml NS (1:1,000; 0.1 ml/kg). The apparatus will then communicate: “Stop CPR and give five assisted ventilations”. Clock C is then set for 3 minutes.

In pediatric step 5 (VT/VF) the apparatus communicates: “Continue CPR at 100 compressions per minute” and the apparatus will provide a metronomic cadence at 100 beats per minute. Once clock B reaches 2 minutes, the apparatus communicates: “Stop CPR/Check rhythm”. Three options are then presented: VT/VF (continue below), Asystole (go to pediatric step 18), Organized Rhythm (go to pediatric step 28(b)). If the first option is chosen, the apparatus communicates: “Defibrillate at 4 joules per kilogram” and “All Clear” (three times) and “Continue CPR at 100 compressions per minute”. The metronomic cadence commences at 100 beats per minute. Clock B is set at 2 minutes and restarted.

In pediatric step 6 (with IV/IO access) (VT/VF) the apparatus communicates medicine, dosage and method of administration: Amiodarone 5 mg/kg IV/IO or Lidocaine 1 mg/kg IV/IO. Clock D is set for 3 minutes. Alternatively, in pediatric step 6(a), without IV/IO access, but with airway device confirmed, the apparatus will communicate: Lidocaine 2-3 mg/kg ET in 5 ml water or NS. The apparatus will then communicate: “Stop CPR and give five assisted ventilations”. Clock D is then set for 3 minutes. The apparatus can note that Amiodarone can not be given through an airway device, thus it is not provided as an option. Once it is indicated that an IV is established, the apparatus will no longer communicate a need to perform pediatric step 4(a) or 6(a) during the remainder of the protocols.

In pediatric step 7, (VT/VF) clock B reaches 2 minutes and the apparatus communicates: “Stop CPR/Check rhythm”. Three options are then presented: VT/VF (continue below), Asystole (go to pediatric step 18), Organized Rhythm (go to pediatric step 28(b)). If the first option is chosen, the apparatus communicates: “Defibrillate at 4 joules per kilogram” and “All Clear” (three times) and “Continue CPR at 100 compressions per minute”. The metronomic cadence commences at 100 beats per minute. Clock B is set at 2 minutes and restarted.

In pediatric step 8 (with IV/IO access) (VT/VF) the apparatus communicates medicine, dosage and method of administration: Epinephrine 0.01 mg IV/IO (1:10,000; 0.1 ml/kg). Clock C is set for 3 minutes. Alternatively, in pediatric step 8(a), without IV/IO access, but with airway device confirmed, the apparatus will communicate: Epinephrine 0.1 mg/kg ET in 5 ml NS (1:1,000; 0.1 ml/kg). The apparatus will then communicate: “Stop CPR and give five assisted ventilations”. Clock C is then set for 3 minutes.

In pediatric step 9 (VT/VF) clock B reaches 2 minutes and the apparatus communicates: “Stop CPR/Check rhythm”. Three options are then presented: VT/VF (continue below), Asystole (go to pediatric step 18), Organized Rhythm (go to pediatric step 28(b)). If the first option is chosen, the apparatus communicates: “Defibrillate at 4 joules per kilogram” and “All Clear” (three times) and “Continue CPR at 100 compressions per minute”. The metronomic cadence commences at 100 beats per minute. Clock B is set at 2 minutes and restarted.

In pediatric step 10 (with IV/IO access) (VT/VF) the apparatus communicates medicine, dosage and method of administration: Lidocaine 1 mg/kg IV/. Clock D is set for 3 minutes. Alternatively, in pediatric step 10(a), without IV/IO access, but with airway device confirmed, the apparatus will communicate: Lidocaine 2-3 mg/kg ET in 5 ml water or NS. The apparatus will then communicate: “Stop CPR and give five assisted ventilations”. Clock D is then set for 3 minutes. The apparatus will then communicate “Check D-stick”.

In pediatric step 11 (VT/VF) clock B reaches 2 minutes and the apparatus communicates: “Stop CPR/Check rhythm”. Three options are then presented: VT/VF (continue below), Asystole (go to pediatric step 18), Organized Rhythm (go to pediatric step 28(b)). If the first option is chosen, the apparatus communicates: “Defibrillate at 4 joules per kilogram” and “All Clear” (three times) and “Continue CPR at 100 compressions per minute”. The metronomic cadence commences at 100 beats per minute. Clock B is set at 2 minutes and restarted.

In pediatric step 12 (with IV/IO access) (VT/VF) the apparatus communicates medicine, dosage and method of administration: Epinephrine 0.01 mg IV/IO (1:10,000; 0.1 ml/kg). Clock C is set for 3 minutes. Alternatively, in pediatric step 12(a), without IV/IO access, but with airway device confirmed, the apparatus will communicate: Epinephrine 0.1 mg/kg ET in 5 ml NS (1:1,000; 0.1 ml/kg). The apparatus will then communicate: “Stop CPR and give five assisted ventilations”. Clock C is then set for 3 minutes.

In pediatric step 13 (VT/VF) clock B reaches 2 minutes and the apparatus communicates: “Stop CPR/Check rhythm”. Three options are then presented: VT/VF (continue below), Asystole (go to pediatric step 18), Organized Rhythm (go to pediatric step 28(c)). If the first option is chosen, the apparatus communicates: “Defibrillate at 4 joules per kilogram” and “All Clear” (three times) and “Continue CPR at 100 compressions per minute”. The metronomic cadence commences at 100 beats per minute. Clock B is set at 2 minutes and restarted.

In pediatric step 14 (VT/VF) the apparatus communicates: “Has the patient received the maximum dose of 100 mg of Lidocaine?” If the response is no, then the apparatus communicates, in pediatric step 14(a): Lidocaine 1 mg/kg IV/IO and the clock is no longer set. Alternatively, the apparatus continues to pediatric step 14(b). If the response is yes, then the apparatus continues to pediatric step 15. In pediatric step 14(b), without IV access, but with airway device confirmed, the apparatus communicates: Lidocaine 2-3 mg/kg ET in 5 ml water or NS. The apparatus will then communicate: “Stop CPR and give five assisted ventilations”. The clock is no longer set.

In pediatric step 15 the apparatus communicates: “Have you considered the following reversible causes?” The following prompts are then communicated: Hypovolemia, Drug overdose, Hypoxia, Cardiac tamponade, Acidosis, Tension pneumothorax, Hyperkalemia, Pulmonary embolism, Hypokalemia, Coronary thrombosis, Hypothermia. The user is then given the option to save the list for later reference or hide the list. The apparatus may include a prompt button or for this list on the screen at this time so if it needs to be referenced again the user has easy access to it.

In pediatric step 16 (VT/VF) clock B reaches 2 minutes and the apparatus communicates: “Stop CPR/Check rhythm”. Three options are then presented: VT/VF (continue below), Asystole (go to pediatric step 18), Organized Rhythm (go to pediatric step 28(c)). If the first option is chosen, the apparatus communicates: “Defibrillate at 4 joules per kilogram” and “All Clear” (three times) and “Continue CPR at 100 compressions per minute”. The metronomic cadence commences at 100 beats per minute. Clock B is set at 2 minutes and restarted.

In pediatric step 17 (with IV/IO access) (VT/VF) the apparatus communicates medicine, dosage and method of administration: Epinephrine 0.01 mg IV/IO (1:10,000; 0.1 ml/kg). Clock C is set for 3 minutes. Alternatively, in pediatric step 17(a), without IV/IO access, but with airway device confirmed, the apparatus will communicate: Epinephrine 0.1 mg/kg ET in 5 ml NS (1:1,000; 0.1 ml/kg). The apparatus will then communicate: “Stop CPR and give five assisted ventilations”. Clock C is then set for 3 minutes. At this point in the method, the apparatus performs a nested loop of pediatric steps 16 and 17. There are no new medications until the team is ready to “call the code” and pronounce the patient dead. At that point, the termination sequence stays on screen until pediatric step 54.

In pediatric step 18 (Asystole/PEA) the apparatus communicates: “Begin CPR at 100 compressions per minute”. Clock B is then set for 2 minutes and started. The apparatus then prompts: “Is IV/IO access established?” in accordance with pediatric step 3. If not, the prompt is continued and asked again every five minutes until an affirmative response is provided. Meanwhile, the apparatus communicates: “Is airway device in place and confirmed?” The prompt is continued and asked again every five minutes until an affirmative response is provided. The prompt could remain on the touch screen until the IV/IO and/or an airway is established.

In pediatric step 19 (with IV/IO access) (Asystole/PEA) the apparatus communicates medicine, dosage and method of administration: Epinephrine 0.01 mg IV/IO (1:10,000; 0.1 ml/kg). Clock C is set for 3 minutes. Alternatively, in pediatric step 19(a), without IV/IO access, but with airway device confirmed, the apparatus will communicate: Epinephrine 0.1 mg/kg ET in 5 ml NS (1:1,000; 0.1 ml/kg). The apparatus will then communicate: “Stop CPR and give five assisted ventilations”. Clock C is then set for 3 minutes.

In pediatric step 20 (Asystole/PEA) clock B reaches 2 minutes and the apparatus communicates: “Stop CPR/Check rhythm”. Three options are then presented: VT/VF (go to pediatric step 2), Asystole (continue below), Organized Rhythm (go to pediatric step 28(b)). The apparatus communicates: “Continue CPR at 100 compressions per minute”. The metronomic cadence commences at 100 beats per minute. Clock B is set at 2 minutes and restarted.

In pediatric step 21 (with IV/IO access) (Asystole/PEA) the apparatus communicates medicine, dosage and method of administration: Epinephrine 0.01 mg IV/IO (1:10,000; 0.1 ml/kg). Clock C is set for 3 minutes. Alternatively, in pediatric step 21(a), without IV/IO access, but with airway device confirmed, the apparatus will communicate: Epinephrine 0.1 mg/kg ET in 5 ml NS (1:1,000; 0.1 ml/kg). The apparatus will then communicate: “Stop CPR and give five assisted ventilations”. Clock C is then set for 3 minutes.

In pediatric step 22 (Asystole/PEA) the apparatus communicates: “Have you considered the following reversible causes?” The following prompts are then communicated: Hypovolemia, Drug overdose, Hypoxia, Cardiac tamponade, Acidosis, Tension pneumothorax, Hyperkalemia, Pulmonary embolism, Hypokalemia, Coronary thrombosis, Hypothermia. The user is then given the option to save the list for later reference or hide the list. The apparatus may include a prompt button or for this list on the screen at this time so if it needs to be referenced again the user has easy access to it.

In pediatric step 23 (Asystole/PEA) clock B reaches 2 minutes and the apparatus communicates: “Stop CPR/Check rhythm”. Three options are then presented: VT/VF (go to pediatric step 2), Asystole (continue below), Organized Rhythm (go to pediatric step 28(b)). The apparatus communicates: “Continue CPR at 100 compressions per minute”. The metronomic cadence commences at 100 beats per minute. Clock B is set at 2 minutes and restarted.

In pediatric step 24 (with IV/IO access) (Asystole/PEA) the apparatus communicates medicine, dosage and method of administration: Epinephrine 0.01 mg IV/IO (1:10,000; 0.1 ml/kg). Clock C is set for 3 minutes. Alternatively, in pediatric step 24(a), without IV/IO access, but with airway device confirmed, the apparatus will communicate: Epinephrine 0.1 mg/kg ET in 5 ml NS (1:1,000; 0.1 ml/kg). The apparatus will then communicate: “Stop CPR and give five assisted ventilations”. Clock C is then set for 3 minutes.

In pediatric step 25 (Asystole/PEA) the apparatus communicates: “Stop CPR/Check rhythm”. Three options are then presented: VT/VF (go to pediatric step 2), Asystole (continue below), Organized Rhythm (go to pediatric step 28(c)). The apparatus communicates: “Continue CPR at 100 compressions per minute”. The metronomic cadence commences at 100 beats per minute. Clock B is set at 2 minutes and restarted.

In pediatric step 26 (Asystole/PEA) the apparatus communicates: “Have you considered the following reversible causes?” The following prompts are then communicated: Hypovolemia, Drug overdose, Hypoxia, Cardiac tamponade, Acidosis, Tension pneumothorax, Hyperkalemia, Pulmonary embolism, Hypokalemia, Coronary thrombosis, Hypothermia. The user is then given the option to save the list for later reference or hide the list.

In pediatric step 27 (Asystole/PEA) the apparatus communicates medicine, dosage and method of administration: Epinephrine 0.01 mg IV/IO (1:10,000; 0.1 ml/kg). Clock C is set for 3 minutes. Alternatively, in pediatric step 26(a), without IV/IO access, but with airway device confirmed, the apparatus will communicate: Epinephrine 0.1 mg/kg ET in 5 ml NS (1:1,000; 0.1 ml/kg). The apparatus will then communicate: “Stop CPR and give five assisted ventilations”. Clock C is then set for 3 minutes. At this point in the method, the apparatus performs a nested loop of pediatric steps 25 to 27. Pediatric step 28(c) creates the loop. There are no new medications until the team is ready to “call the code” and pronounce the patient dead. At that point, the termination sequence stays on screen until pediatric step 54.

Pediatric steps 28(a)-(c) comprise pediatric step 28 (Organized Rhythm). In pediatric step 28(a), the apparatus communicates “Check Pulse.” If a pulse is present, then the apparatus goes to pediatric step 29. If there is no pulse, the apparatus goes to pediatric step 21. In pediatric step 28(b), the apparatus communicates “Check Pulse.” If a pulse is present, then the apparatus goes to pediatric step 29. If there is no pulse, the apparatus goes to pediatric step 24. In pediatric step 28(c), the apparatus communicates “Check Pulse.” If a pulse is present, then the apparatus goes to pediatric step 29. If there is no pulse, the apparatus goes to pediatric step 27.

In pediatric step 29 (post-resuscitative care/decompensated), the apparatus communicates: “Check vital signs” and “Consider a fluid bolus” (10-20 mL/kg of NS or RL). The apparatus then prompts: Hypotensive or Decompensated (continue below) or Normotensive or compensated (go to pediatric step 30). The apparatus then prompts: Consider further fluid boluses; Consider: Oxygen, labs, CXR, pain control, NG-tube, secure IV lines, pulse oximeter and monitor; Epinephrine drip at 0.1 to 1 mcg/kg min; and/or Dopamine drip 2 to 20 mcg/kg/min; and/or Norepinephrine drip 0.1 to 2 mcg/kg/min.

In pediatric step 30 (Post-resuscitative care/compensated), the apparatus prompts: Consider further fluid boluses; Consider: Oxygen, labs, CXR, pain control, NG-tube, secure IV lines, pulse oximeter and monitor; Dobutamine drip at 2 to 20 mcg/kg/min; and/or Dopamine drip 2 to 20 mcg/kg/min and/or Epinephrine drip 0.05 to 0.3 mcg/kg/min and/or Milrinone drip: Load with 50 to 75 mcg/kg. Infuse at 0.5 to 0.75 mcg/kg min

In pediatric step 31 (Pulse present), the apparatus communicates: “Check vital signs” and “Give oxygen” and “Is IV or 10 access established?” (see pediatric step 3). If the answer is no, the question is re-asked every three minutes, or left on the display screen until affirmative.

In pediatric step 32 (Pulse present), three options are presented: Bradycardia (go to pediatric step 33), Normal rate (continue below) and Tachycardia (go to pediatric step _). The apparatus communicates: “Is the heart rate causing cardiopulmonary compromise?” If yes, continue to pediatric step 33. If not, the apparatus communicates: “Monitor and observe”.

In pediatric step 33 (Pulse present/bradycardia), the apparatus communicates: “Heart rate less than 60 or is the patient unstable?” If yes, the apparatus goes to pediatric step 34. If no, the apparatus communicates: “Monitor, ventilate and observe”.

In pediatric step 34, the apparatus communicates: “Is there poor perfusion despite proper ventilation?” If yes, go to pediatric step 34(a), if not, the apparatus communicates: “Monitor, ventilate and observe”. In pediatric step 34(a) (unstable bradycardia) the apparatus communicates: “Continue CPR at 100 compressions per minute”. The metronomic cadence commences at 100 beats per minute. Clock B is set at 2 minutes and restarted.

In pediatric step 35 (with IV/IO access) (Unstable bradycardia) the apparatus communicates medicine, dosage and method of administration: Epinephrine 0.01 mg IV/IO (1:10,000; 0.1 ml/kg). Clock C is set for 3 minutes. Alternatively, in pediatric step 35(a), without IV/IO access, but with airway device confirmed, the apparatus communicates: Epinephrine 0.1 mg/kg ET in 5 ml NS (1:1,000; 0.1 ml/kg). The apparatus will then communicate: “Stop CPR and give five assisted ventilations”. Clock C is then set for 3 minutes. The apparatus communicates: “Check D-stick”.

In pediatric step 36 (Unstable bradycardia) the apparatus communicates: “Have you considered the following reversible causes?” The following prompts are then communicated: Hypovolemia, Drug overdose, Hypoxia, Cardiac tamponade, Acidosis, Tension pneumothorax, Hyperkalemia, Pulmonary embolism, Hypokalemia, Coronary thrombosis, Hypothermia. The user is then given the option to save the list for later reference or hide the list. The apparatus may include a prompt button or for this list on the screen at this time so if it needs to be referenced again the user has easy access to it.

In pediatric step 37 (Unstable bradycardia) clock B reaches 2 minutes and the apparatus communicates: “Stop CPR/Check rhythm”. Three options are then presented: VT/VF (go to pediatric step 2), Asystole (go to pediatric step 18), Organized Rhythm (go to pediatric step 28(b)). The apparatus communicates: “Continue CPR at 100 compressions per minute”. The metronomic cadence commences at 100 beats per minute. Clock B is set at 2 minutes and restarted.

In pediatric step 38 (Unstable bradycardia) the apparatus communicates medicine, dosage and method of administration: Epinephrine 0.01 mg IV/IO (1:10,000; 0.1 ml/kg). Clock C is set for 3 minutes. Alternatively, in pediatric step 38(a), without IV/IO access, but with airway device confirmed, the apparatus communicates: Epinephrine 0.1 mg/kg ET in 5 ml NS (1:1,000; 0.1 ml/kg). The apparatus will then communicate: “Stop CPR and give five assisted ventilations”. Clock C is then set for 3 minutes.

In pediatric step 39 (Tachycardia), the apparatus communicates: “Is the heart rate causing cardiopulmonary compromise or poor profusion?” If yes, go to pediatric step 43. If no, the apparatus prompts: QRS duration greater than 0.08 second? If yes, go to pediatric step 40. If not, then the apparatus communicates: “History compatible with sinus tachycardia or P waves present?” If yes, go to pediatric step 42. If no, go to pediatric step 41.

In pediatric step 40, (Wide-complex tachycardia/stable), the apparatus communicates: “Possible ventricular tachycardia” and provides the following prompts: Consult pediatric cardiologist. Consider: Support ABC's with oxygen, IV line, pulse oximeter, monitor and attach defibrillator pads; Amiodarone 5 mg/kg IV over 20 to 60 minutes or Procainamide 15 mg/kg IV over 30 to 60 minutes or Lidocaine 1 mg/kg IV bolus. If patient becomes unstable shock at 0.5 to 1 joule/kg. May increase to 2 joules/kg if initial shock is ineffective.

In pediatric step 41 (Narrow-complex tachycardia/stable), the apparatus communicates: “Possible SVT”. The apparatus prompts: Consult pediatric cardiologist. Consider Vagal maneuvers; Adenosine 0.1 mg/kg IV rapid bolus (max 1st dose 6 mg); May double and repeat dose once (max 2nd dose 12 mg); Support ABC's with oxygen, IV line, pulse oximeter, monitor and defibrillator pads

In pediatric step 42 (Narrow complex tachycardia/stable), the apparatus communicates: “Probable sinus tachycardia”. The apparatus prompts: Consult pediatric cardiologist. Consider: Support ABC's with oxygen, IV line, pulse oximeter, monitor and defibrillator pads; May attempt fluid bolus; Search for etiologies

In pediatric step 43 (Narrow-complex tachycardia/unstable) the apparatus prompts: QRS duration greater than 0.08 second? If yes, go to pediatric step 45. If not, then the apparatus communicates: “History compatible with sinus tachycardia or P waves present?” If no, go to pediatric step 44. If yes, the apparatus communicates: “Probable sinus tachycardia”. The apparatus prompts: Consult pediatric cardiologist. Consider: Support ABC's with oxygen, IV line, pulse oximeter, monitor and attach defibrillator pads; May attempt fluid bolus; Search for etiologies.

In pediatric step 44 (Narrow-complex tachycardia/unstable), the apparatus communicates: “Possible SVT”. The apparatus prompts: Consult pediatric cardiologist. Consider sedation then the apparatus communicates: “Cardiovert at 0.5 to 1 Joule/kg”. The apparatus prompts: May increase to 2 joules/kg if initial shock ineffective. Consider Vagal maneuvers; Adenosine 0.1 mg/kg IV rapid bolus (max 1st dose 6 mg); May double and repeat dose once (max 2nd dose 12 mg); Support ABC's with oxygen, IV line, pulse oximeter, monitor and defibrillator pads.

In pediatric step 45 (Wide-complex tachycardia/unstable), the apparatus communicates: “Possible ventricular tachycardia” The apparatus prompts: Consider sedation then the apparatus communicates: “Cardiovert at 0.5 to 1 Joule/kg”. The apparatus prompts: May increase to 2 joules/kg if initial shock ineffective. Consider Amiodorone 5 mg/kg IV over 20 to 60 minutes, or Procainamide 15 mg/kg IV over 30 to 60 minutes or Lidocaine 1 mg/kg IV bolus. Consult pediatric cardiologist. Consider: Support ABC's with oxygen, IV line, pulse oximeter, monitor

In pediatric step 54 the termination sequence is invoked. The apparatus communicates: “Are you sure you want to end ACLS activities?” If yes, then the apparatus communicates: “Confirm you want to end ACLS activities?” If no, the loop continues. At this point, the user is given an option to print a summary of events with the timing of drugs given relative to the external clocks and the internal elapsed time clock. In one aspect of the invention, a sheet of paper having an adhesive back that can be inserted into the apparatus, printed on and then ejected from the apparatus.

The apparatus shall be adapted to provide audible and visual commands, prompts and reminders in a variety of languages. An embodiment of the present invention includes a touch sensitive display providing navigation and selection of on-screen, graphical input elements, Input buttons providing program path/option selection, internal timer providing user prompts and program path selection. Further, an embodiment of the apparatus of the present invention is adapted to output the audio commands using speakers or other sound transducers. The clocks that are presented to the responder can include lighted, segmented displays providing numeric time representation (digital clock) adapted to count-down, count-up, and timing for user actions within the program. Processing of the various commands within the CPU of the computer hardware includes customary computer operations. Subsequent actions within the program perform timing, prompting, and/or subsequent input functions. Subsequent prompts are displayed on the graphical display and are announced via the audio output. Timing operations (count-down1 count-up, time elapsed, and absolute time) are performed by the program, and output via the segmented display. Subsequent user input for path selection, acknowledgement of user prompts within the program is accomplished via the input buttons and I or touch display.

The application software in which a method of the present invention is embodied is adapted to be stored within a computer hardware memory device, including a hard drive, RAM memory, or standard media, such as memory cards, flash memory devices, magnetic disks, optical disks, or other commonly used storage media. Program updates, including updates to ACLS algorithms, can be effected by over-writing obsolete code with new program files and/or program data on the memory media.

The form factor housing an embodiment of the present invention can include a rectangular cube housing, having front perforations with a speaker mounted within the housing behind the perforations, and aperture for receiving a touch sensitive graphical display, buttons protruding from the top near the front and segmented display protrudes from the top, facing front, near the back.

The embodiments shown and described above are only exemplary. Even though numerous characteristics and advantages of the preferred embodiment of the present invention have been set forth in the foregoing description together with details of the invention, the disclosure is illustrative only and changes may be made within the principles of the invention to the full extent indicated by the broad general meaning of the terms used in the attached claims.

I claim:

1. An apparatus for use in performing Advanced Cardiac Life Support (ACLS) procedures, comprising:
at least one prompting means for obtaining information from a user;
at least one input means for obtaining user responses to information prompts;
at least one timing means for obtaining timing information based elapsed time of the ACLS, or events occurring during the ACLS process;
logic means operable to accept user information and timing information and determine and output a decision path selection based thereon; and
at least one output means for communicating instructions to guide users through the proper procedures to be applied to a patient during a cardiac arrest based on output from the logic means.

2. The apparatus of claim 1, wherein the at least one timing means and logic means are implemented using computer hardware having an operating system, and application software, the computer hardware further comprising a power supply, an input means, data busses, a central processing unit, a memory unit, an output means.

3. The apparatus of claim 2, the computer hardware being located in a stand-alone unit adapted to be installed on a crash cart.

4. The apparatus of claim 2, the hardware being one selected from the group consisting of a mainframe, personal computer (PC), terminal, tablet PC, laptop PC and personal digital assistant (PDA).

5. The apparatus of claim 2, wherein the input means consists of one selected from the group consisting of a keyboard, mouse, touchpad, trackball and touch screen display; and
the output means consists of one selected from the group consisting of a speaker, buzzer, illuminated indicator, video display and printer.

6. The apparatus of claim 1, further comprising:
the logic means including a computerized decision tree program adapted to be run on computer hardware;
a look-up table within a computerized memory means adapted to store information to be conveyed to the output means based on input to the input means and timing information;
a memory means adapted to store digital representations of video and audio command and prompt data;
a digital to analog converter and voice synthesizer adapted to convert digital audio data to analog signals; and
the output means comprising a sound transducer for outputting the analog voice signals that guide responders through the proper procedures to be applied to a patient during a cardiac arrest.

7. The apparatus of claim 6, wherein the analog voice signals are in one from the group selected from the following languages consisting of English, Spanish, German, French, Chinese, Japanese, Russian, Portuguese, Korean, Vietnamese, Swedish, Norwegian, and Finnish.

8. The apparatus of claim 6, wherein the software is adapted to receive software updates.

9. The apparatus of claim 6, adapted to instruct responders as to the types and dosages of medications to administer, and sequence of performing actions (such as cardiac pulmonary resuscitation (CPR)) on a patient.

10. The apparatus of claim 9, further comprising an external clock adapted to retain the elapsed time from the beginning of the ACLS procedure to the end of the ACLS procedure;
a CPR clock B, adapted to track an individual CPR session;
a Vasopressor clock C, adapted to track the timing of this class of medications (Epinephrine or Vasopressin);
an Antiarrythmic clock D, adapted to track the timing of Epinephrine or Amiodarone;
an Atropine clock E, adapted to track the timing of Atropine and
an IV/Airway clock.

11. The apparatus of claim 1, in combination with an automatic external defibrillator (AED).

12. The apparatus of claim 1, adapted to be configured to communicate a first set of instructions if the patient is an adult and a second set of instructions if the patient is a child.

13. The apparatus of claim 1, adapted to give medicine type and dosage instructions by referencing the medicines as contained in coded packaging maintained with the apparatus.

14. The apparatus of claim 1, adapted to output, via a video display or printer, a list of actions taken during the ACLS process, correlated to the actual time such events occurred.

15. A system for providing real-time instruction on the use of equipment and the procedures to be followed in the resuscitation of a patient, said system comprising:
an input means and output means for providing an array of inputs related to program path and option selection;
at least one timer providing user prompts and program path selection;
an array of outputs, including:
voice prompts of user actions and tuning; graphic display of user information; digital time display;
a logic unit adapted to operate on information from the timer and the inputs to direct and prompt users during resuscitation of a patient experiencing cardiac distress.

16. The apparatus of claim 14, wherein the input/output means comprises a touch sensitive display screen.

17. The apparatus of claim 14, the system comprising computer hardware selected from the group consisting of a mainframe, personal computer (PC), terminal, tablet PC, laptop PC and personal digital assistant (PDA);
an operating system; and
application software adapted to implement the processes of the system

18. The system of claim 14, in combination with an automatic external defibrillator (AED).

19. The system of claim 14, adapted to be configured to communicate a first set of instructions if the patient is an adult and a second set of instructions if the patient is a child.

20. A method for providing real-time instruction on the use of equipment and the procedures to be followed in the resuscitation of a patient, said system comprising:
providing an input means and output means for providing an array of inputs related to program path and option selection;
obtaining information on whether the patient is an adult or child;
based on the information, selecting, by the logic unit, either a first set of instructions or a second set of instructions.
providing at least one timer to time the provision of user prompts and program path selection;
providing an array of outputs; and
operating, by a logic unit, on information from the timer and the inputs to direct and prompt users during resuscitation of a patient experiencing cardiac distress.
